SEQUENCE LISTING (12 sequences)

SEQ ID NO: 1 - DNA, 20 bp, Artificial Sequence
  Synthetic Polynucleotide
ctgcgacttcaacagcaact

SEQ ID NO: 2 - DNA, 20 bp, Artificial Sequence
  Synthetic Polynucleotide
gagttgggatagggcctctc

SEQ ID NO: 3 - DNA, 23 bp, Artificial Sequence
  Synthetic Polynucleotide
ctgcatcttggactctaatggac

SEQ ID NO: 4 - DNA, 23 bp, Artificial Sequence
  Synthetic Polynucleotide
gcgatattacttttctccgtgtg

SEQ ID NO: 5 - DNA, 23 bp, Artificial Sequence
  Synthetic Polynucleotide
caaagtatagccacctgttcacc

SEQ ID NO: 6 - DNA, 23 bp, Artificial Sequence
  Synthetic Polynucleotide
gatcttttggggctgtagtttct

SEQ ID NO: 7 - DNA, 22 bp, Artificial Sequence
  Synthetic Polynucleotide
atcacgttcaaggtcaccctac

SEQ ID NO: 8 - DNA, 22 bp, Artificial Sequence
  Synthetic Polynucleotide
ttctgcttctgcctctctctct

SEQ ID NO: 9 - DNA, 20 bp, Artificial Sequence
  Synthetic Polynucleotide
gctcagtcaccctcaggtaa

SEQ ID NO: 10 - DNA, 20 bp, Artificial Sequence
  Synthetic Polynucleotide
aagtttccgccgatgttgga

SEQ ID NO: 11 - DNA, 22 bp, Artificial Sequence
  Synthetic Polynucleotide
ctatattgcgaattacggcaca

SEQ ID NO: 12 - DNA, 22 bp, Artificial Sequence
  Synthetic Polynucleotide
acaccttgcttccattgagaat

(19) United States
(12) Patent Application Publication
(10) Pub. No.: US 2023/0321179 A1
(43) Pub. Date: Oct. 12, 2023

(54) N-ACYL AMINO ACID PRODUCTS AND USES

(71) Applicant: THE REGENTS OF THE UNIVERSITY OF MICHIGAN, Ann Arbor, MI (US)

(72) Inventors: Oren Rom, Ann Arbor, MI (US); Jifeng Zhang, Ann Arbor, MI (US); Yuqing Eugene Chen, Ann Arbor, MI (US); Ying Zhao, Ann Arbor, MI (US)

(21) Appl. No.: 18/020,908

(22) PCT Filed: Aug. 17, 2021
(86) PCT No.: PCT/US2021/046357

§ 371 Date: Feb. 10, 2023

Related U.S. Application Data
(60) Provisional application No. 63/067,175, filed on Aug. 18, 2020.

(51) Int. Cl. A61K 38/06; A61P 1/16; A61K 31/198; A61P 9/10

(52) U.S. Cl. CPC A61K 38/06; A61K 31/198; A61P 1/16; A61P 9/10

(57) ABSTRACT

The present invention relates to N-acyl amino acid products and their use in diagnosing and treating disease.

### INCORPORATION BY REFERENCE OF THE SEQUENCE LISTING

This application contains, as a separate part of disclosure, a Sequence Listing in computer-readable form (filename 55769A_Seqlisting.text; 2,597 bytes - ASCII text file created Aug. 17, 2021) which is incorporated by reference herein in its entirety.

### FIELD OF THE INVENTION

The present invention relates to N-acyl amino acid products and their use in diagnosing and treating disease.

### BACKGROUND

Cardiovascular disease (CVD) arising from atherosclerosis is a leading cause of death worldwide. Non-alcoholic fatty liver disease (NAFLD), the most common chronic liver disease, precedes and/or promotes the development of atherosclerosis. A subset of NAFLD patients develops a more severe non-alcoholic steatohepatitis (NASH) and liver fibrosis, which can further accelerate atherosclerosis progression and CVD events. Indeed, CVD is a major cause of death in NAFLD patients, particularly those with NASH.

There remains a need in the art for products and methods for diagnosing and treating NASH, fibrosis and CVD.

### SUMMARY

Abnormal lipid metabolism is a hallmark feature of both CVD and NAFLD. It is contemplated herein that dysregulated metabolism of specific amino acids also plays a role in the pathogenesis of CVD and NAFLD. The current application describes the use of N-acyl amino acids, amino acids conjugated to fatty acids, for diagnosis and for the treatment of a subject with one or more of steatohepatitis, fibrosis or a cardiovascular disease condition.

A first aspect herein provides methods for treating a cardiovascular disease condition. The methods comprise administering a therapeutically effective amount of at least one N-acyl amino acid product to a subject with a cardiovascular disease condition. Cardiovascular disease (CVD) conditions involve the heart and blood vessels, and they include coronary heart disease, cerebrovascular disease, peripheral arterial disease, rheumatic heart disease, congenital heart disease, aortic aneurysm, and deep vein thrombosis and pulmonary embolism.

A second aspect herein provides methods for reducing fibrosis. The methods comprise administering a therapeutically effective amount of at least one N-acyl amino acid product to a subject with fibrosis.

A third aspect herein provides methods of treating steatohepatitis. The methods comprise administering a therapeutically effective amount of at least one N-acyl amino acid product to a subject with steatohepatitis.

A fourth aspect herein provides N-acyl amino acid products and compositions, including pharmaceutical compositions. Exemplary N-acyl amino acid products include, but are not limited to, N-acyl glycine, N-acyl leucine, N-acyl-D-leucine, N-acyl glycine-glycine-leucine, N-acyl glycine-glycine-D-leucine, their pharmaceutical salts, or a combination of at least two thereof.

A fifth aspect herein provides methods of diagnosing a disease condition such as a cardiovascular disease condition, fibrosis or steatohepatitis. The methods comprise detecting N-acyl amino acids, for example, N-acyl glycine, N-acyl leucine and/or N-acyl-D-leucine.

### BRIEF DESCRIPTION OF THE DRAWINGS

FIG. 1. Suppressed metabolism and levels of N-acyl amino acids in NASH. pPCR analysis of hepatic expression of (A) Pm20d1 and (B) Glyat. LC-MS/MS analysis of hepatic concentrations of (C) N-oleoyl glycine (C:18-Gly), (D) N-arachidonoyl glycine (C20:4-Gly) and (E) N-oleoyl leucine (C18:1-Leu). Data are mean±SEM (n=8). *P<0.05; * P<0.01; ***P<0.001 vs. CD; #P<0.05, ##P<0.01 ###P<0.001 vs. NASH +H2O.

FIGS. 2A-2D. Correlation between hepatic levels of N-acyl amino acids and NASH severity. Spearman correlation was calculated between hepatic levels of N-oleoyl glycine (C181-Gly), N-arachidonoyl glycine (C20:4-Gly) and N-oleoyl leucine (C18:1-Leu) and (A) hepatic steatosis assessed by lipid extraction and TG quantification, (B) inflammatory infiltration assessed by F4/80 positive area, (C) fibrosis score assessed by Sirius Red staining and (D) NAFLD activity score (NAS) in mice (n=8-9) fed CD (■) or NASH diet and treated with H2O (control, ▲), leucine (▼), glycine (✦), tripepetide glycine-glycine-leucine (0.125 mg/g/d, o) or tripepetide glycine-glycine-leucine (0.5 mg/g/d, •).

FIGS. 3A-3C. Correlation between hepatic levels of N-acyl amino acids and circulating cardiometabolic risk factors. Spearman correlation was calculated between hepatic levels of N-oleoyl glycine (C18:1-Gly), N-arachidonoyl glycine (C20:4-Gly) and N-oleoyl leucine (C18:1-Leu) and plasma levels of (A) ALT, (B) MCP-1 and (C) TC in mice (n=8-9) fed CD (■) or NASH diet and treated with H2O(control, A), leucine (▼), glycine (✦), tripepetide glycine-glycine-leucine (0.125 mg/g/d, o) or tripepetide glycine-glycine-leucine (0.5 mg/g/d, •).

FIG. 4. N-acyl amino acids directly activate PPARα. (A, B) COS-1 cells were co-transfected with PPREx3-TK-luciferase, PPARα and Renilla. 24 h post-transfection, cells were treated with 10 µM of the PPARα agonist WY-14643, 1 mM of glycine or tripepetide glycine-glycine-leucine or 10 µM of N-oleoyl glycine (C18:1-Gly), N-arachidonoyl glycine (C20:4-Gly) or N-oleoyl leucine (C18:1-Leu) for 24 h. Luciferase activity was normalized by Renilla. ***P<0.001 vs. CTL.

FIGS. 5A-5C. Correlation between hepatic levels of N-acyl amino acids and the expression of PPARα target genes. Spearman correlation was calculated between hepatic levels of N-oleoyl glycine (C18:1-Gly), N-arachidonoyl glycine (C20:4-Gly) and N-oleoyl leucine (C18:1-Leu) and the expression of (A) Ppargc1a, (B) Acot3 and (C) Acad/ in mice (n=8-9) fed CD (■) or NASH diet and treated with H2O(control, ▲), leucine (▼), glycine (✦), tripepetide glycine-glycine-leucine (0.125 mg/g/d, o) or tripepetide glycine-glycine-leucine (0.5 mg/g/d, •).

FIGS. 6A-6D. N-acyl amino acids stimulate lipid utilization via FAO. (A, B) Oxygen consumption rate (OCR) and dependency on FAO assessed using a Seahorse XFe96 Analyzer. HepG2 were stimulated with 10 µM of N-arachidonoyl glycine (C20:4-Gly), N-oleoyl leucine (C18:1-Leu) or vehicle (ethanol, EtOH) and then treated with 6 µM of the CPT1 inhibitor, etomoxir (n=8). (C, D) Lipid biosynthesis and hydrolysis rates assessed by monitoring the incorporation of [3H]-acetate (3.3 µCi/ml) into TG in HepG2 cells treated with 10 µM of N-arachidonoyl glycine (C20:4-Gly), N-oleoyl leucine (C18:1-Leu) or vehicle (ethanol, EtOH) (n=6).

FIG. 7. Experimental design of a NASH study in mice.

FIGS. 8A-8C. N-oleoyl leucine (C18:1-Leu) lowers body weight without affecting adiposity. NMR-based body composition analysis at weeks 21 to 22 (n=8): (A) Body weight, (B) fat (%), and (C) lean mass (%). Data are means ± SEM. Statistical differences were compared by one-way ANOVA followed by Tukey post hoc test or by Kruskal-Wallis test followed by Dunn’s post hoc test. *** P<0.001 vs. SD; ### P<0.001 vs. NASH; AAA P<0.001 vs. NASH +C18:1.

FIGS. 9A-9D N-oleoyl leucine (C18:1-Leu) has no significant effect on systemic energy balance in NASH. Metabolic parameters were assessed using a comprehensive laboratory animal monitoring system (CLAMS) at weeks 21 to 22 (n=8): (A) respiratory exchange ratio (RER), (B) energy expenditure (EE), (C) food intake, and (D) total activity. Data are means ± SEM. Statistical differences were compared by one-way ANOVA followed by Tukey post hoc test or by Kruskal-Wallis test followed by Dunn’s post hoc-test. * P<0.05, ** P<0.01, *** P<0.001 vs. SD.

FIGS. 10A-10B. N-oleoyl leucine (C18:1-Leu) significantly lowers hepatomegaly. (A) Gross morphology of the liver, and (B) liver weight to body weight ratio at endpoint (n=8-10). Data are means ± SEM. Statistical differences were compared by Kruskal-Wallis test followed by Dunn’s post hoc-test. *** P<0.001 vs. SD, ## P<0.01 vs. NASH; ^ P<0.05 vs. NASH +C18:1.

FIGS. 11A-11C. N-oleoyl leucine (C18:1-Leu) lowers circulating liver enzymes. Circulating liver enzymes at endpoint: (A) alanine-aminotransferase (ALT), (B) aspartate aminotransferase (ASP), and (C) alkaline phosphatase (ALP) (n=8-10).. Data are means ± SEM. Statistical differences were compared by one-way ANOVA followed by Tukey post hoc test or by Kruskal-Wallis test followed by Dunn’s post hoc-test. ** P<0.01, *** P<0.001 vs. SD; # P<0.05, ## P<0.01 vs. NASH.

FIGS. 12A-12D. N-oleoyl leucine (C18:1-Leu) significantly lowers diet-induced NASH. (A) Hematoxylin and eosin (H&E) histology of the liver (scale bar = 50 µm). (B-E) H&E histology was used to score (B) hepatic steatosis (0-3), (C) lobular inflammation (0-3), and (D) hepatocyte ballooning (0-2). NAFLD activity score (NAS) was calculated as the sum of the above scores (n=8-10). Data are means ± SEM. Statistical differences were compared by Kruskal-Wallis test followed by Dunn’s post hoc-test. *** P<0.001 vs. SD, # P<0.05 vs. NASH; ^ P<0.05 vs. NASH +C18:1.

FIGS. 13A-13B. N-oleoyl leucine (C18:1-Leu) significantly lowers hepatic steatosis. (A) Oil red O (ORO) histology of the liver (scale bar = 100 µm). (B) Plasma total cholesterol (TC) (n=8-10). Data are means ± SEM. Statistical differences were compared by Kruskal-Wallis test followed by Dunn’s post hoc-test. ** P<0.01, *** P<0.001 vs. SD.

FIGS. 14A-14C. N-oleoyl leucine (C18:1-Leu) significantly lowers NASH diet-induced hepatic and systemic inflammation. (A) F4/80 immunohistochemistry of the liver (scale bar = 50 µm). (B) Plasma C-C motif chemokine ligand 2 (CCL2), and (C) CCL5 (n=8-10). Data are means ± SEM. Statistical differences were compared by one-way ANOVA followed by Tukey post hoc or by Kruskal-Wallis test followed by Dunn’s post hoc-test. ** P<0.01, *** P<0.001 vs. SD., # P<0.05 vs. NASH.

FIGS. 15A-15B. N-oleoyl leucine (C18:1-Leu) significantly lowers NASH diet-induced hepatic fibrosis. (A) Sirius red histology of the liver (scale bar = 50 µm). (B) Fibrosis score based on Sirius red histology (n=8-10). Data are means ± SEM. Statistical differences were compared by Kruskal-Wallis test followed by Dunn’s post hoc-test. ** P<0.01, *** P<0.001 vs. SD., # P<0.05 vs. NASH.

FIG. 16. Experimental design of an atherosclerosis study in mice.

FIGS. 17A-17B. N-oleoyl leucine (C18:1-Leu) treatment has no significant effects on body weight and plasma cholesterol in atherosclerotic mice. (A) Body weight, and (B) plasma total cholesterol (TC) at endpoint. Data are means ± SEM. Statistical differences were compared by Unpaired t test.

FIG. 18. N-oleoyl leucine (C18:1-Leu) significantly lowers atherosclerosis. H&E histology of the aortic sinus was used to quantify the plaque area. Data are means ± SEM. Statistical differences were compared by Mann Whitney test. * P<0.05.

FIG. 19. N-oleoyl leucine (C18:1-Leu) significantly lowers lesional macrophages. Mac-2 immunohistochemistry of the aortic sinus was used to quantify the content of lesional macrophages. Data are means ± SEM. Statistical differences were compared by Mann Whitney test. ** P<0.01.

### DETAILED DESCRIPTION

N-acyl amino acid products are products in which the acyl moiety of a long chain fatty acid is covalently linked to an amino acid.

The amino acid component of an N-acyl amino acid product herein can be glycine or leucine, or a peptide comprising glycine and leucine. The peptide can be, for example, a dipeptide or tripeptide. With the exception of glycine, the common amino acids all contain at least one chiral carbon atom. Leucine exists in two forms, stereoisomers designated as the L-isomer and the D-isomer. Most naturally occurring proteins and peptides are composed exclusively of the L-isomeric form. Leucine-containing N-acyl amino acid products herein comprise L-leucine unless D-leucine is specified.

Exemplary dipeptide amino acid components are glycine-glycine, glycine-leucine, glycine-D-leucine, leucine-leucine, D-leucine-leucine, D-leucine-D-leucine, and leucine-D-leucine.

Exemplary tripeptide amino acid components are glycine-glycine-leucine and glycine-glycine-D-leucine.

The long chain fatty acid component of an N-acyl amino acid product herein can be a polyunsaturated fatty acid or a nitro fatty acid.

An exemplary N-acyl amino acid product is N-palmitoyl glycine.

An exemplary N-acyl amino acid product is N-stearoyl glycine.

An exemplary N-acyl amino acid product is N-oleoyl glycine.

An exemplary N-acyl amino acid product is N-docosahexaenoyl glycine.

An exemplary N-acyl amino acid product is N-arachidonoyl glycine.

An exemplary N-acyl amino acid product is N-palmitoyl leucine.

An exemplary N-acyl amino acid product is N-stearoyl leucine.

An exemplary N-acyl amino acid product is N-oleoyl leucine.

An exemplary N-acyl amino acid product is N-docosahexaenoyl leucine.

An exemplary N-acyl amino acid product is N-arachidonoyl leucine.

An exemplary N-acyl amino acid product is N-palmitoyl D-leucine.

An exemplary N-acyl amino acid product is N-stearoyl D-leucine.

An exemplary N-acyl amino acid product is N-oleoyl D-leucine.

An exemplary N-acyl amino acid product is N-docosahexaenoyl D-leucine.

An exemplary N-acyl amino acid product is N-arachidonoyl D-leucine.

An exemplary N-acyl amino acid product is N-palmitoyl glycine-glycine-leucine.

An exemplary N-acyl amino acid product is N-stearoyl glycine-glycine-leucine.

An exemplary N-acyl amino acid product is N-oleoyl glycine-glycine-leucine.

An exemplary N-acyl amino acid product is N-docosahexaenoyl glycine-glycine-leucine.

An exemplary N-acyl amino acid product is N-arachidonoyl glycine-glycine-leucine.

An exemplary N-acyl amino acid product is N-palmitoyl glycine-glycine-D-leucine.

An exemplary N-acyl amino acid product is N-stearoyl glycine-glycine-D-leucine.

An exemplary N-acyl amino acid product is N-oleoyl glycine-glycine-D-leucine.

An exemplary N-acyl amino acid product is N-docosahexaenoyl glycine-glycine-D-leucine.

An exemplary N-acyl amino acid product is N-arachidonoyl glycine-glycine-D-leucine.

The fatty acid component of an N-acyl amino acid product herein can be a polyunsaturated fatty acid (PUFA) such as a linoleic acid, a conjugated linoleic acid or an omega 3 fatty acid. Exemplary omega 3 fatty acids include, but are not limited to, docosahexaenoic acid, α-linolenic acid or eicosapentanoic acid. The fatty acid component of an N-acyl amino acid product herein can be a metabolite of an omega 3 fatty acid such as a furan fatty acid or a resolvin. An exemplary furan fatty acid is 3-carboxy-4-methyl-5-propyl-2-furanpropanoic acid. An exemplary resolvin is Resolvin D.

The fatty acid component of an N-acyl amino acid product herein can be a nitro-fatty acid such as 10-nitro-octadec-9-enoic acid, 9-nitro-octadec-9-enoic acid, a nitrated ω-3 fatty acid (including, but not limited to, linolenic acid, alphalinolenic acid, eicosapentanoic acid, docosapentaenoic acid, docosahexanoic acid and stearidonic acid), a nitrated ω-5 fatty acid (including, but not limited to, myristoleic acid), a nitrated ω-6 fatty acid (including, but not limited to, linoleic acid, gamma-linoleic acid, dihomo-gamma-linoleic acid and arachidonic acid), a nitrated ω-7 fatty acid (including, but not limited to, conjugated linoleic and palmitoleic acid) or a nitrated ω-9 fatty acid (including, but not limited to, oleic acid and erucic acid).

Combinations of different N-acyl amino acid products are also provided. For example, combinations of two or more of N-arachidonoyl glycine, N-oleoyl leucine and N-oleoyl D-leucine are provided. As yet another example, combinations of N-arachidonoyl glycine and N-oleoyl leucine are provided. As another example, combinations of two or more of N-arachidonoyl glycine-glycine-leucine, N-oleoyl glycine-glycine-leucine, N-arachidonoyl glycine-glycine-D-leucine and N-oleoyl glycine-glycine-D-leucine are provided. As yet a further example, combinations of two or more of N-arachidonoyl glycine, N-oleoyl leucine, N-arachidonoyl glycine-glycine-leucine, N-oleoyl glycine-glycine-leucine, N-arachidonoyl glycine-glycine-D-leucine and N-oleoyl glycine-glycine-D-leucine are provided.

N-acyl amino acid products herein also include pharmaceutically acceptable salts. Pharmaceutically acceptable salts are well known in the art. For example, Berge et al. describe pharmaceutically acceptable salts in detail in J. Pharmaceutical Sciences, 66: 1-19 (1977). Examples of such salts include metal salts, ammonium salts, salts with organic base, salts with inorganic acid, salts with organic acid, salts with basic or acidic amino acid, and the like. Examples of a metal salt include alkali metal salts such as sodium salt, potassium salt and the like; alkaline earth metal salts such as calcium salt, magnesium salt, barium salt and the like; aluminum salt and the like. Examples of a salt with organic base include salts with trimethylamine, triethylamine, pyridine, picoline, 2,6-lutidine, ethanolamine, diethanolamine, triethanolamine, cyclohexylamine, dicyclohexylamine, N,N-dibenzylethylenediamine and the like. Examples of a salt with inorganic acid include salts with hydrochloric acid, hydrobromic acid, nitric acid, sulfuric acid, phosphoric acid and the like. Examples of a salt with organic acid include salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid and the like.

N-acyl amino acid products herein, or a pharmaceutically acceptable salt thereof, can be synthesized and/or administered as prodrugs of their original synthetic forms. A prodrug is a compound which is converted to the product described herein by a reaction due to an enzyme, gastric acid, etc. under the physiological condition in the living body, that is, a compound which is converted to the glycine tripeptide molecule or a pharmaceutically acceptable salt thereof, with oxidation, reduction, hydrolysis, etc. according to an enzyme; a compound which is converted to the glycine tripeptide molecule by hydrolysis etc. due to gastric acid, etc. See, for example, IYAKUHIN no KAIHATSU (Development of Pharmaceuticals), Vol.7, Design of Molecules, p.163-198, Published by HIROKAWA SHOTEN (1990).

The peptide component of an N-acyl amino acid product herein can be produced by peptide synthesis methods known in the art. A peptide synthesis method may employ condensation reactions, for example, in a solid phase synthesis method or a liquid phase synthesis method. If the product produced has a protecting group, the protecting group is removed. Examples of known peptide synthesis methods include methods described in the following: M. Bodanszky and M.A. Ondetti: Peptide Synthesis, Interscience Publishers, New York (1966); Schroeder and Luebke: The Peptide, Academic Press, New York (1965); Nobuo Izumiya, et al.: Peptide Gosei-no-Kiso to Jikken (Basics and experiments of peptide synthesis), published by Maruzen Co. (1975); Haruaki Yajima and Shunpei Sakakibara: Seikagaku Jikken Koza (Biochemical Experiment) 1, Tanpakushitsu no Kagaku (Chemistry of Proteins) IV, 205 (1977); and Haruaki Yajima, ed.: Zoku lyakuhin no Kaihatsu (A sequel to Development of Pharmaceuticals), Vol. 14, Peptide Synthesis, published by Hirokawa Shoten.

Compositions provided herein comprise at least one N-acyl amino acid product, or comprise combinations of N-acyl amino acid products.

Pharmaceutical compositions provided herein comprise a pharmaceutically acceptable excipient, and at least one N-acyl amino acid product or a combination of two or more N-acyl amino acid products.

Pharmaceutical compositions suitable for the delivery of N-acyl amino acid products herein and methods for their preparation are readily apparent to those skilled in the art. Remington’s Pharmaceutical Sciences, The Science and Practice of Pharmacy, 22nd Edition, Lippincott Williams & White, Baltimore, MD (2013) provides exemplary standard considerations and methods.

Pharmaceutical compositions herein are formulated with pharmaceutically acceptable excipients such as carriers, solvents, stabilizers, adjuvants, diluents, etc., depending upon the particular mode of administration and dosage form. Pharmaceutical composition components can be included for modifying, maintaining or preserving, for example, the pH, osmolarity, viscosity, clarity, color, isotonicity, odor, sterility, stability, rate of dissolution or release, adsorption or penetration of the composition. The compositions are generally formulated to achieve a physiologically compatible pH, and range from a pH of about 3 to a pH of about 11, about pH 3 to about pH 7, or about pH 5.0 to about pH 8, depending on the formulation and route of administration.

Suitable excipients include, for example, sterile liquids, such as water and oils, including those of petroleum, animal, vegetable or synthetic origin, such as peanut oil, soybean oil, mineral oil, sesame oil, and the like. Water is a typical excipient when the pharmaceutical composition is administered intravenously. Saline solutions (including, but not limited to, a sodium chloride solution) and aqueous dextrose and glycerol solutions can be employed as liquid excipients, particularly for injectable solutions. Additional suitable pharmaceutical excipients include starch, glucose, lactose, sucrose, gelatin, malt, rich, flour, chalk, silica gel, sodium stearate, glycerol monostearate, talc, sodium chloride, dried skim milk, glycerol, propylene, glycol, water, ethanol, and the like. A larger list of excipients contemplated includes, but is not limited to, amino acids (such as glycine, glutamine, asparagine, arginine or lysine); antimicrobials; antioxidants (such as ascorbic acid, sodium sulfite or sodium hydrogen sulfite); buffers (such as borate, bicarbonate, Tris HCI, citrates, phosphates, other organic acids); bulking agents (such as mannitol or glycine), chelating agents (such as ethylenediamine tetraacetic acid (EDTA)); complexing agents (such as caffeine, polyvinylpyrrolidone, beta cyclodextrin or hydroxypropyl beta cyclodextrin); fillers; monosaccharides; disaccharides and other carbohydrates (such as glucose, mannose, hydroxyalkylcellulose, hydroxyalkylmethylcellulose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (in one aspect, sodium or potassium chloride, mannitol sorbitol); delivery vehicles; diluents; and/or carrier molecules that include large, slowly metabolized macromolecules such as proteins, polysaccharides, polylactic acids, polyglycolic acids, polymeric amino acids, amino acid copolymers, and inactive virus particles.

Pharmaceutical compositions herein can take the form of solutions, suspensions, emulsions, tablets, pills, capsules, powders, sustained-release formulations, and the like. Pharmaceutical compositions herein may be formulated for immediate and/or modified release of an N-acyl amino acid product.

Pharmaceutical compositions herein are administered by any suitable route, for example, by an intravenous, oral, ocular, intradermal, subcutaneous, intraperitoneal or intramuscular route. It is contemplated that administration by the oral route is accomplished using delivery vehicles known in the art which would minimize degradation of N-acyl amino acids in the gastrointestinal tract including, but not limited to, microspheres, liposomes, enteric-coated dry emulsions, tablets, or nanoparticles.

Kits for administering an N-acyl amino acid product or combinations of products to a subject in need thereof, comprise an N-acyl amino acid product composition described herein, instructions for use of the N-acyl amino acid product composition, and optionally an additional second therapeutic agent or therapy.

An exemplary stock composition herein comprises N-arachidonoyl glycine and/or n-oleoyl leucine diluted in 100% ethanol for a final concentration of 50 mg/ml. N-arachidonoyl glycine is stored at -80° C. and n-oleoyl leucine is stored at -20° C. An exemplary pharmaceutical composition herein is then freshly prepared from the stock composition by dilution in a mixture of 100% ethanol and sterile saline solution (0.9% NaCl) at a ratio of 4:15:81 (v:v:v), to yield of a composition of 2 mg/ml concentration of N-acyl amino acid product.

An exemplary stock composition herein comprising N-acyl glycine-glycine-leucine and/or N-acyl glycine-glycine-D-leucine comprises a lyophilized cake, prepared in a formulation buffer consisting of 10 mM glutamic acid, 2% glycine, 1% sucrose, and 0.01% polysorbate 20 to pH 4.25. An exemplary pharmaceutical composition herein is then prepared by reconstitution with a volume of sterile diluent, for example, sterile isotonic saline or water, for example, 0.5 mL to about 10 mL, for example, 2.2 mL of sterile water, to yield composition of 1 g/mL to about 100 g/mL concentration of N-acyl amino acid product.

Methods are provided of administering to a subject a pharmaceutical composition comprising a therapeutically effective amount of an N-acyl amino acid product or combination of products described herein.

A subject may be a mammal and the mammal may be, for example, a laboratory animal or a human, and human subjects include adult, adolescent and pediatric subjects.

A “therapeutically effective amount” as used herein refers to an amount of N-acyl amino acid product sufficient to exhibit a detectable therapeutic effect. The effect is detected by an improvement in clinical condition, and/or a reduction, elimination or inhibition of development of a particular symptom or event associated with the condition. The precise effective amount for a subject will depend upon the subject’s body weight, size, and health; the nature and extent of the condition; and the product or combination of products selected for administration. Therapeutically effective amounts are determined by routine experimentation that is within the skill and judgment of the clinician.

A pharmaceutical composition herein can be administered to a subject by any suitable route as noted above. For example, compositions of the invention can be administered by an intravenous, oral, ocular, intradermal, intraperitoneal, subcutaneous or intramuscular route.

One skilled in the art appreciates that the effective amount varies depending, in part, upon the molecule delivered, the indication for which the composition is being used, the route of administration, and the size (body weight, body surface or organ size) and condition (the age and general health) of the subject. Accordingly, the clinician may titer a dose and modify the route of administration to obtain the optimal therapeutic effect. A therapeutically effective amount can be a dose including, but not limited to, from about 1 mg/kg to about 10,000 mg/kg, from about 1 mg/kg to about 1,000 mg/kg, from about 0.1 mg/kg to about 1,000 mg/kg, from about l mg/kg to about 1,000 mg/kg, from about 1,000 mg/kg to about 10,000 mg/kg, or from about 1 mg/kg to about 500 mg/kg, calculated on the subject’s bodyweight. An exemplary therapeutically effective dose is from about 100 mg to about 200 g. An exemplary therapeutically effective dose is from about 100 mg/kg to about 200 mg/kg. Another exemplary therapeutically effective dose is from about 0.01 mg/kg to about 200 mg/kg. Another exemplary therapeutically effective dose is from about 10 mg/kg to about 200 mg/kg. Another exemplary therapeutically effective dose is from about 1 mg to about 10 mg. A dose can be given daily, two or three times daily, every other day, twice weekly, weekly, monthly, or semi-annually. Delivery can also be by continuous infusion. Methods described herein can be used for treating, for example, cardiovascular disease conditions, steatohepatitis and fibrosis.

The term “treating” (or other forms of the word such as “treatment” or “treat”) is used herein to mean that administration of a composition of the present invention mitigates a condition in a subject and/or reduces, inhibits, or eliminates a particular symptom or event associated with a condition. Thus, the term “treatment” includes, preventing a condition from occurring in a subject, particularly when the subject is predisposed to acquiring the condition; reducing or inhibiting the condition; and/or ameliorating or reversing the condition. Insofar as the methods of the present invention are directed to preventing conditions, it is understood that the term “prevent” does not require that the condition be completely avoided.

Cardiovascular disease conditions are disease conditions of the heart and blood vessels including, but not limited to: coronary heart disease — disease of the blood vessels supplying the heart muscle; cerebrovascular disease — disease of the blood vessels supplying the brain; peripheral arterial disease — disease of blood vessels supplying the arms and legs; rheumatic heart disease — damage to the heart muscle and heart valves from rheumatic fever, caused by streptococcal bacteria; congenital heart disease — malformations of heart structure existing at birth; aortic aneurysm - an abnormal bulge in the wall of the aorta; and deep vein thrombosis and pulmonary embolism — blood clots in the leg veins, which can dislodge and move to the heart and lungs. Treatment of cardiovascular disease conditions results in one or more of the following mitigations detectable by standard techniques, including but not limited to: reduction in atherosclerotic plaques (e.g., demonstrated by ultrasound imaging), increase in cardiac function, reduction in myocardial hypertrophy [e.g., demonstrated by ultrasound imaging, computed tomography scan, magnetic resonance imaging, or analysis of biomarkers such as troponin and/or BMP (or other biomarkers such as those listed at page e101 of Tang et al., Circulation, 116: e99-e109 (2007))], decrease in blood pressure, decrease in inflammatory status (e.g., demonstrated by analysis of circulating inflammatory markers such as MCP-1, C-reactive protein, serum amyloid A protein, heat shock protein 65, interleukin-6 and leukocyte adhesion molecules), and decrease in aortic diameter.

Events associated with cardiovascular disease conditions that are mitigated by treatment herein include, but are not limited to, heart failure, decompensation (e.g., of the heart or liver), myocardial infarction and aneurysms.

Steatohepatitis is a type of fatty liver disease, characterized by inflammation of the liver with concurrent fat accumulation in the liver. Non-alcoholic steatohepatitis (NASH) damage to the liver is similar to the damage seen in steatohepatitis caused by heavy alcohol use. Macro and microscopically, NASH is characterized by lobular and/or portal inflammation, varying degrees of fibrosis, hepatocyte death and pathological angiogenesis. At its most severe, NASH can progress to cirrhosis, hepatocellular carcinoma and liver failure. Steatohepatitis treatment methods herein can be monitored by a subject’s NAFLD Activity score. NAFLD Activity score (NAS) can be calculated according to the criteria of Kleiner et al., Hepatology, 41:1313-1321 (2005). NAS scores 0-2 are not considered diagnostic for NASH, NAS scores of 3-4 are considered either not diagnostic, borderline or positive for NASH, while NAS scores of 5-8 are largely considered diagnostic for NASH. Sequential liver biopsies from a subject that may have NASH can be used to assess the change in the NAS score and used as an indication of the change in the disease state. A score that increases suggests progression, an unchanged score suggests stabilization, while a decreased score suggests regression of NASH. Treatment of steatohepatitis herein results in one or more mitigations detectable by standard techniques including, but not limited to: decrease in liver fat (e.g., demonstrated by lipid staining such as with Oil Red O, biochemical analysis of triglycerides, or ultrasound imaging), decrease in inflammatory status (e.g., demonstrated by histology such as referred to in Table 1 of Kleiner, supra, or analysis of circulating inflammatory markers such as MCP-1, C-reactive protein, serum amyloid A protein, heat shock protein 65, interleukin-6 and leukocyte adhesion molecules), reduction in injured hepatocytes (e.g., demonstrated by histology) and reduction in atherosclerotic plaques (e.g., demonstrated by ultrasound imaging).

Fibrosis is pathological wound healing in which connective tissue replaces normal parenchymal tissue to the extent that it leads to extensive tissue remodeling and the formation of permanent scar tissue. Excessive accumulation of extracellular matrix components, such as collagen produced by fibroblasts, leads to the formation of a permanent fibrotic scar. Fibrosis is scored from 0-4 (0: no fibrosis; 1: perisinusoidal or portal fibrosis; 2: perisinusoidal and portal fibrosis; 3: bridging fibrosis; 4: cirrhosis). See, for example, Table 1 of Kleiner, supra. A score that increases suggests progression, an unchanged score suggests stabilization, while a decreased score suggests regression of fibrosis. Treatment of fibrosis herein results in a reduction in fibrosis detectable by standard techniques in one or more of the liver, heart, lungs, kidneys, skin and adipose tissue. Treatment of fibrosis herein can result in a reduction in fibrosis detectable by standard techniques in one or more of the bile duct, gallbladder, or other structures involved in the production and transportation of bile. Collagen accumulation, for example, is routinely detected by staining such as with Picrosirius Red or Masson’s Trichrome, or by detection of hydroxyproline.

Treatment herein can include treatment with one or more N-acyl amino acid products in combination with a second therapeutic agent such as other lipid- and/or glucose-lowering agents. Other lipid- and/or glucose-lowering agents include, but not limited to, statins, fibrates, SGLT2i, metformin and incretins.

Diagnostic methods herein comprise detecting N-acyl amino acids, for example, N-acyl glycine, N-acyl leucine and/or N-acyl-D-leucine in a subject. Diagnosis herein is contemplated to include initial diagnosis and/or monitoring the state of progression/regression of a disease condition. The hepatic levels of such N-acyl amino acids are negatively associated with the severity of hepatic steatosis, fibrosis, inflammation and hypercholesterolemia.

### EXAMPLES

The present invention is illustrated by the following examples which include a long-term dietary model of NASH featuring coexistence of steatohepatitis and fibrosis in mice. An unbiased analysis of hepatic gene expression by RNA-sequencing followed by qPCR validation, revealed that genes encoding for enzymes that catalyze the condensation of fatty acids and various amino acids (peptidase M20 domain containing 1, Pm20d1) and particularly with glycine (glycine-N-acyltransferase, Glyat), were suppressed in NASH. Targeted metabolomics showed that the levels of N-oleoyl glycine (C18:1-Gly), N-arachidonoyl glycine (C20:4-Gly) and N-oleoyl leucine (C18:1-Leu) were significantly decreased in livers from mice with NASH. This reduction was rescued by chronic treatment with free glycine or with a tri-peptide glycine-glycine-leucine. The hepatic levels of the above N-acyl amino acids were significantly and negatively associated with the severity of hepatic steatosis, fibrosis, inflammation as well as hypercholesterolemia, while positively associated with the expression of target genes of the master regulator of fatty acid β-oxidation (FAO), peroxisome proliferator-activated receptor-α (PPARα). Applying Seahorse and luciferase assays, N-acyl amino acids were found to directly activate PPARα, stimulate mitochondrial respiration and FAO. In conclusion, N-acyl amino acids mediate hepatic lipid utilization and improve energy metabolism, and thus constitute an effective therapeutic approach against CVD, steatohepatitis and fibrosis.

### Example 1

### The Tripepetide Glycine-Glycine-Leucine Protects Against NASH by Regulating Liver Metabolism and Levels of N-acyl Amino Acids

To explore the therapeutic potential of tripepetide glycine-glycine-leucine against NASH, an experimental approach to model advanced NAFLD was used. As described, C57BL/6J mice were fed a high-fat, high-fructose and high-cholesterol diet (NASH diet) for 12 weeks. After confirming NASH, the mice were randomized to receive orally tripepetide glycine-glycine-leucine at 0.125 or 0.5 mg/g/day, equivalent amounts of leucine, glycine or H2O for 12 additional weeks on NASH diet. Mice fed low-fat control diet (CD) and administered H2O served as control.

### Methods

### Animals

Animal procedures were approved (PRO00008239) by the Institutional Animal Care & Use Committee of the University of Michigan (U-M) and performed in accordance with the institutional guidelines. Seven weeks-old male C57BL/were from Jackson Laboratories. After one week of acclimation, mice were fed ad libitum either low-fat control diet (CD, Research Diets D17072805, 10% fat) or high-fat, high-fructose and high-cholesterol diet (NASH diet, Research Diets D17010103). After confirming NASH, the mice were randomized to receive orally tripepetide glycine-glycine-leucine (Beijing SL Pharmaceutical) at 0.125 or 0.5 mg/g/day, equivalent amounts of leucine (0.17 mg/g/day, Sigma-AldrichL8912), glycine (0.33 mg/g/day, Sigma-Aldrich G5417) or H2O for 12 additional weeks on NASH diet.

### Histology and Immunohistochemistry

All histological procedures were performed by the In Vivo Animal Core (IVAC) Histology Laboratory at the U-M. Technicians were blinded to experimental groups. Formalin-fixed tissues were processed through graded alcohols and cleared with xylene followed by infiltration with molten paraffin using an automated VIP5 or VIP6 tissue processor (TissueTek, Sakura-Americas). Using a Histostar Embedding Station (ThermoFisher Scientific), tissues were then sectioned on a M355S rotary microtome (ThermoFisher Scientific) at 4 µm thickness and mounted on glass slides. Slides were stained for hematoxylin and eosin (H&E, ThermoFisher Scientific). For Sirius Red staining, slides were treated with 0.2 phosphomolybdic acid for 3 min and transferred to 0.1% Sirius Red saturated in picric acid (Rowley Biochemical Inc.) for 90 min, then transferred to 0.01N hydrochloric acid for 3 min.

Frozen section processing was used for Oil Red O staining. Formalin-fixed liver samples were cryoprotected in 20% sucrose at 4° C. overnight, blotted, then liquid nitrogen-snap frozen in OCT compound (Tissue-Tek, Cat #4583) and stored at -80° C. until ready for cryosectioning. Prior to sectioning, frozen blocks were brought up to about -20° C., then sectioned at 5 µm on a Cryotome SME (Thermo-Shandon, Cat# 77200227). Slides were stored at -80° C. until stained. Prior to staining, liver slides were thawed to room temperature for 30 min. Slides were post-fixed in 10% Neutral Buffered Formalin for 20 min, rinsed in DDW, followed by rinsing in 60% isopropanol before being placed in working Oil Red O-isopropanol stain (Rowley Biochemical Inc., H-503-1B) for 5 min. Slides were then rinsed in 60% isopropanol followed by three changes of DDW. Then, slides were nuclear counterstained in Harris Hematoxylin and mounted in Aqua-Mount (Lerner Laboratories, Cat# 13800) aqueous mounting media.

Immunohistochemical staining was performed on a IntelliPATH FLX automated immunohistochemical stainer (Biocare Medical) with blocking for endogenous peroxidases and non-specific binding, followed by detection using a horseradish peroxidase biotin-free polymer based commercial detection system, disclosure with diaminobenzidine chromogen, and nuclear counterstaining with hematoxylin. Specific to F4/80, (Bio-Rad ABD Serotec, Cat# MCA497), the rat monoclonal primary antibody (clone CI:A3-1) was diluted to 1:400 in DaVinci Diluent (Biocare Medical, Cat# PD900) and incubated for 60 min followed by detection using Rat-on-Mouse HRP-Polymer, (Biocare Medical, Cat# RT517) 2-step probe-polymer incubation for 10 and 30 min respectively.

### Scoring of NAFLD Activity and Fibrosis

H&E staining was used to score NAFLD activity score (NAS). Steatosis was scored from 0-3 (0: <5% steatosis; 1: 5-33%; 2: 34-66%; 3: >67%). Hepatocyte ballooning was scored from 0-2 (0: normal hepatocytes, 1: normal-sized with pale cytoplasm, 2: pale and enlarged hepatocytes, at least 2-fold). Lobular inflammation was scored from 0-2 based on foci of inflammation counted at 20X (0: none, 1: <2 foci; 2: ≥2 foci). NAS was calculated as the sum of steatosis, hepatocyte ballooning and lobular inflammation scores. Sirius Red staining was used to score hepatic fibrosis from 0-4 (0: no fibrosis; 1: perisinusoidal or portal fibrosis; 2: perisinusoidal and portal fibrosis; 3: bridging fibrosis; 4: cirrhosis).

### Plasma Analyses

Clinical chemistry assays for ALT and AST were performed by the U-M IVAC on a Liasys 330 chemistry analyzer (AMS Diagnostics) using manufacturer-provided reagents and protocols. Plasma total cholesterol was measured using the Wako Diagnostics kit (999-02601). Plasma MCP-1 was measured using the mouse CCL2/JE/MCP-1 Quantikine ELISA Kit (R&D Systems).

### RNA-Sequencing and Data Analysis

Total RNA from mouse liver samples was extracted using QIAGEN’s RNeasy kit (QIAGEN). Library preparation and sequencing were performed by the U-M DNA Sequencing Core. RNA was assessed for quality using the TapeStation (Agilent, Santa Clara, CA). All samples had RNA integrity numbers (RINs) >8.5. Samples were prepared using the NEBNext Ultra II Directional RNA Library Prep Kit for Illumina (NEB, E7760L) with Poly(A) mRNA Magnetic Isolation Module (NEB, E7490L) and NEBNext Multiplex Oligos for Illumina Unique dual (NEB, E6440L), where 10 ng - 1 µg of total RNA were subjected to mRNA polyA purification. The mRNA was then fragmented and copied into first strand cDNA using reverse transcriptase and dUTP mix. Samples underwent end repair and dA-Tailing step followed by ligation of NEBNext adapters. The products were purified and enriched by PCR to create the final cDNA library. Final libraries were checked for quality and quantity by TapeStation (Agilent) and qPCR using Kapa’s library quantification kit for Illumina Sequencing platforms (Kapa Biosystems, KK4835). Libraries were paired-end sequenced on a NovaSeq 6000 Sequencing System (Illumina).

The quality of the raw FASTQ files was checked through FastQC v0.11.8 (https://www.bioinformatics.babraham.ac.uk/projects/fastqc/). Trimmomatic v.0.35 was used to trim the low-quality reads with the parameters: SLIDINGWINDOW:4:20 MINLEN:25. The resulted high-quality reads were then mapped to the mouse reference genome (GRCm38.90) using HISAT2 v.2.1.0.13. Gene expression quantification was performed using HTSeq-counts v0.6.0 based on the GRCm38.90 genome annotations. The R package DESeq2 was then used to identify significant differentially expressed genes (DEG). We considered genes with adjusted P value less than 0.05 and absolute fold change larger than 2 as significant DEG. The up- and down-regulated DEGs were then analyzed respectively for significantly enriched KEGG pathways using the clusterProfiler package. The significance of the enrichment was determined by right-tailed Fisher’s exact test followed by Benjamini-Hochberg multiple testing adjustment.

### Quantitative Real-time PCR Analysis

Total RNA from mouse liver samples was extracted using QIAGEN’s RNeasy kit (QIAGEN). RNA was reverse-transcribed into cDNA with SuperScript III and random primers (Invitrogen). Specific transcript was assessed by a real-time PCR system (Bio-Rad) using iQ SYBR Green Supermix (Bio-Rad) and the ΔΔCt threshold cycle method of normalization. Gene expression was normalized to Gapdh. Primer pairs used for qPCR were obtained from Integrated DNA Technologies and are listed below:

| | | |
|---|---|---|
| Gapdh | CTGCGACTTCAACAGCAACT (SEQ ID NO: 1) | GAGTTGGGATAGGGCCTCTC (SEQ ID NO: 2) |
| Glyat | CTGCATCTTGGACTCTAATGGAC (SEQ ID NO: 3) | GCGATATTACTTTTCTCCGTGTG (SEQ ID NO: 4) |
| Pm20d1 | CAAAGTATAGCCACCTGTTCACC (SEQ ID NO: 5) | GATCTTTTGGGGCTGTAGTTTCT (SEQ ID NO: 6) |
| Ppargc1a | ATCACGTTCAAGGTCACCCTAC (SEQ ID NO: 7) | TTCTGCTTCTGCCTCTCTCTCT (SEQ ID NO: 8) |
| Acot3 | GCTCAGTCACCCTCAGGTAA (SEQ ID NO: 9) | AAGTTTCCGCCGATGTTGGA (SEQ ID NO: 10) |
| Acadl | CTATATTGCGAATTACGGCACA (SEQ ID NO: 11) | ACACCTTGCTTCCATTGAGAAT (SEQ ID NO: 12) |

### Liver Analysis

Livers were rapidly removed from the euthanized mice, snap-frozen in liquid nitrogen, and kept at -80° C. The LC-MS/MS method for detecting and quantification of N-acyl amino acids was developed by the U-M Pharmacokinetics and Mass Spectrometry Core. Technicians were blinded to experimental groups. The levels of N-acyl amino acids in the liver were normalized to the tissue weight and expressed as ng/g liver tissue. For TG quantification, frozen liver samples (100 mg) were homogenized in PBS and centrifuged (14,000 RPM, 20 min). The supernatants were collected and analyzed for protein concentration using Bio-Rad Bradford assay. To assess liver lipid composition, lipids were extracted from the supernatants using hexane (≥99%, Sigma-Aldrich 32293) and isopropanol (≥99.5%, Fisher Scientific A426-4) at a 3:2 ratio (v:v), and the hexane phase was left to evaporate for 48 h. The amount of liver TG was determined spectrophotometrically using commercially the Wako Diagnostics kit (994-02891).

### Results

At the endpoint, the increase in plasma levels of NAFLD markers, alanine aminotransferase (ALT) and aspartate aminotransferase (AST), was attenuated by glycine or tripepetide glycine-glycine-leucine. Accordingly, NASH diet-induced hepatomegaly was significantly reduced by glycine or tripepetide glycine-glycine-leucine and histological analyses revealed lower hepatic steatosis, inflammation (F4/80 macrophage staining) and fibrosis (Sirius Red staining) with NAFLD activity score (NAS) significantly decreased by 0.5 mg/g/day tripepetide glycine-glycine-leucine.

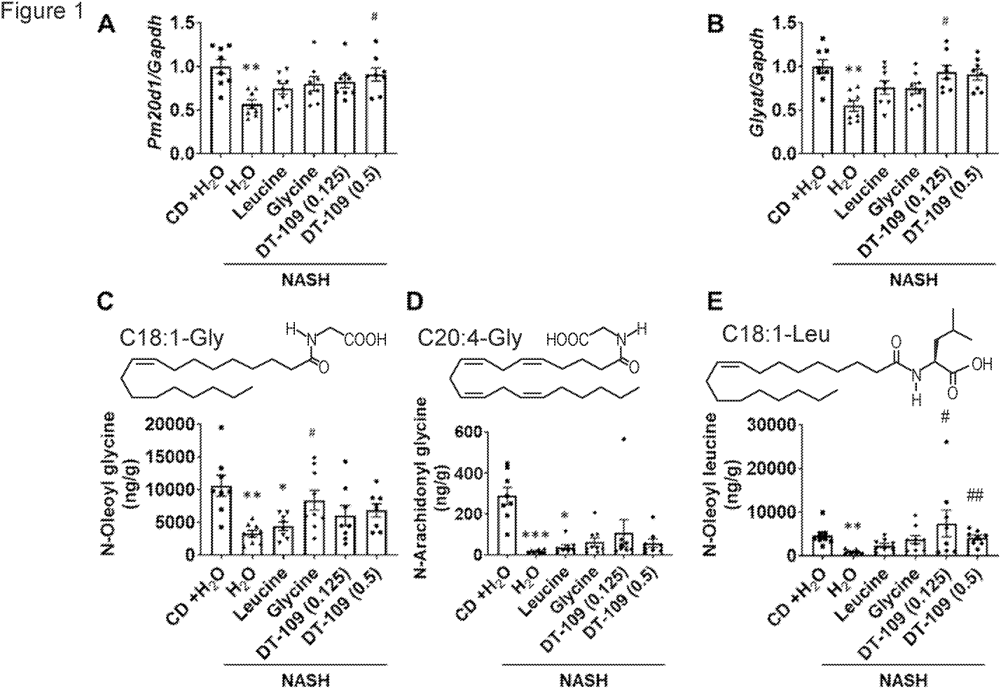

An unbiased analysis of hepatic gene expression by RNA-sequencing revealed that genes encoding for enzymes that catalyze the condensation of fatty acids and various amino acids (peptidase M20 domain containing 1, Pm20d1) and particularly with glycine (glycine-N-acyltransferase, Glyat), were down-regulated in mice with NASH which was reversed by tripepetide glycine-glycine-leucine treatment. These results were confirmed by qPCR analyses (FIGS. 1A, B). Accordingly, targeted metabolomics revealed that the levels of N-oleoyl glycine (C18:1-Gly), N-arachidonoyl glycine (C20:4-Gly) and N-oleoyl leucine (C18:1-Leu) were significantly decreased in livers from mice with NASH which was rescued by chronic treatment with glycine or tripepetide glycine-glycine-leucine (FIGS. 1C-E).

### Example 2

### Hepatic Levels of N-acyl Amino Acids Are Associated With Markers of Steatohepatitis, Fibrosis And Cardiovascular Disease

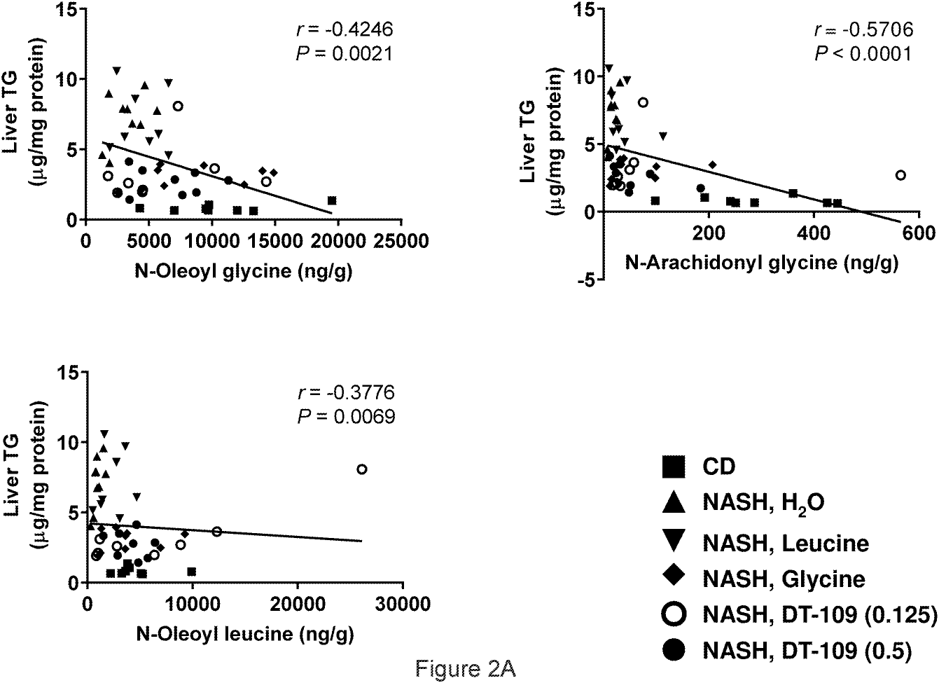

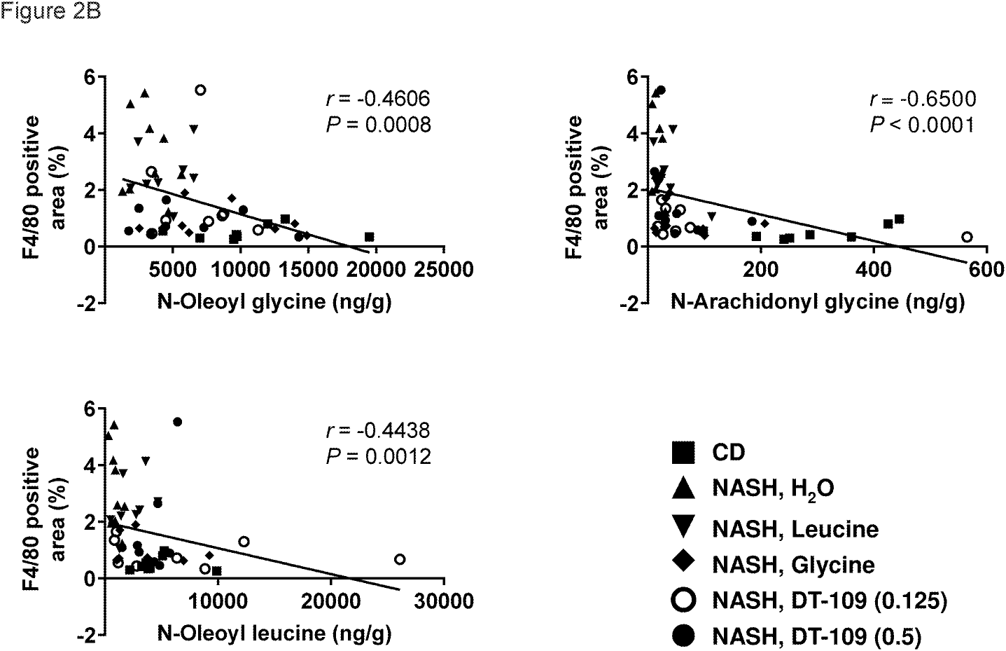

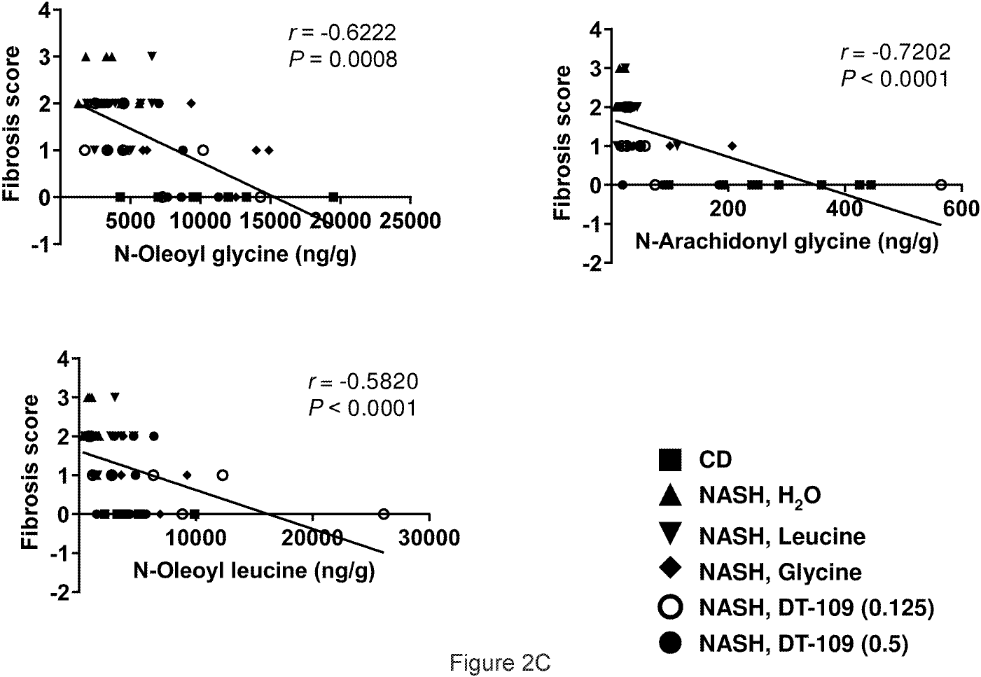

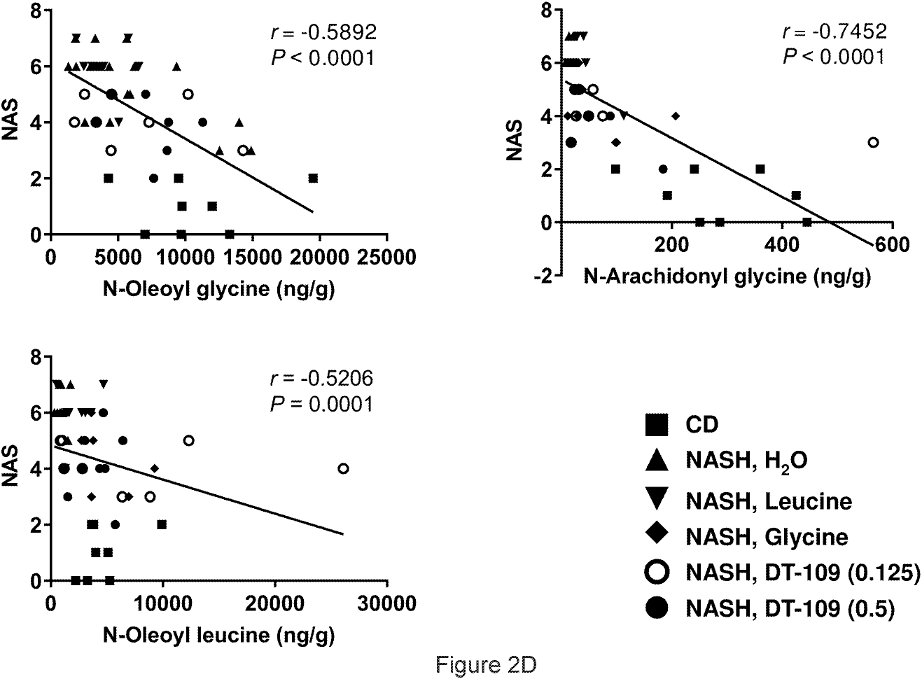

To examine the relationship between N-acyl amino acids and NASH, we analyzed the correlation between the hepatic levels from the mice of Example 1 of N-acyl amino acids and indices of NASH severity, namely, hepatic steatosis (quantification of liver triglycerides, TG), inflammation (F4/80 immunohistochemistry), fibrosis (Sirius Red staining) and overall NAS. We found that the hepatic levels of N-acyl amino acids significantly and inversely correlated with hepatic steatosis (FIG. 2A), inflammation (FIG. 2B), fibrosis (FIG. 2C) and NAS (FIG. 2D). The most significant inverse correlations (P<0.0001) were found between the levels of N-arachidonoyl glycine (C20:4-Gly) and the above indices.

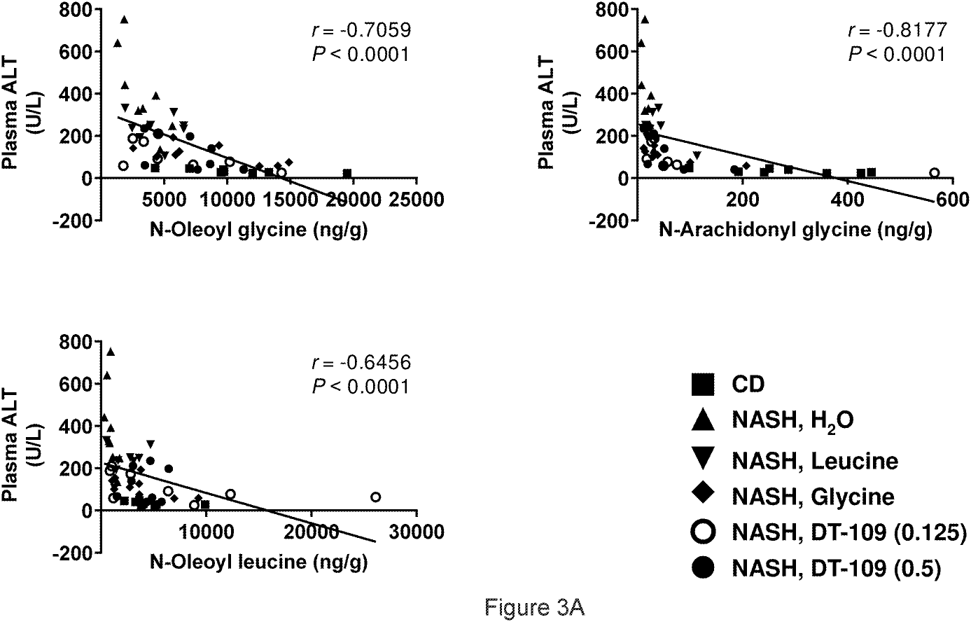

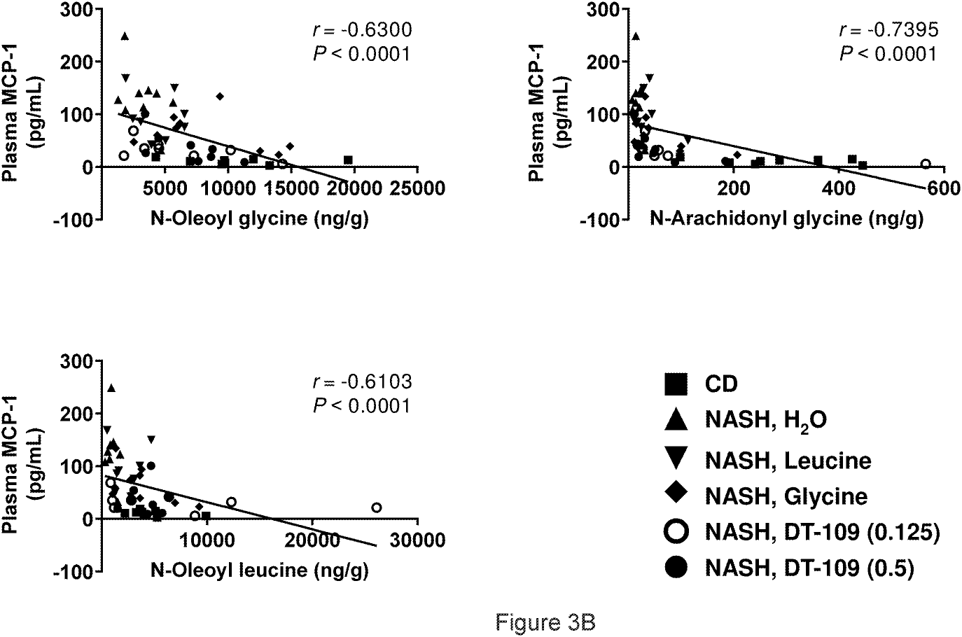

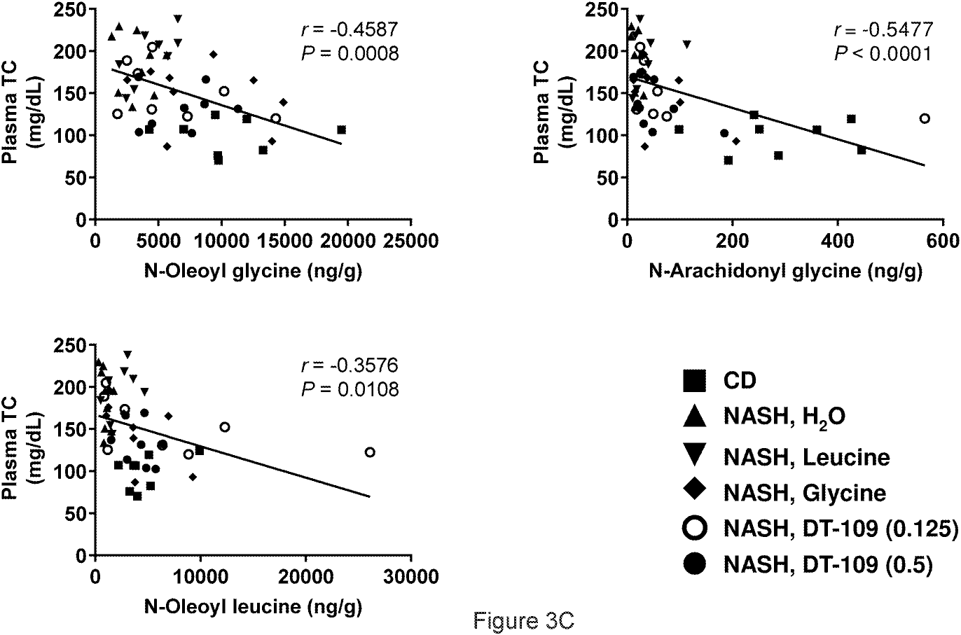

Then, to examine the relationship between N-acyl amino acids and other cardiometabolic risk factors, we analyzed the correlation between the hepatic levels in the mice of Example 1 of N-acyl amino acids and plasma levels of ALT (liver damage marker), monocyte chemoattractant protein 1 (MCP-1, an inflammatory marker) and total cholesterol (TC, one the strongest risk factors of CVD). Similar to the NASH indices (FIGS. 2A-D), hepatic levels of N-acyl amino acids significantly and inversely associated with ALT, MCP-1 and TC, with the most significant correlations (P<0.0001) found for N-arachidonoyl glycine (C20:4-Gly) (FIGS. 3A-C).

### Example 3

### N-acyl Amino Acids Directly Activate PPARα

The unbiased RNA-sequencing analysis described in Example 1 revealed that major pro-inflammatory and pro-fibrotic pathways were enriched in livers from mice with NASH. In contrast, in livers from mice on the NASH diet and treated with tripepetide glycine-glycine-leucine, the most significant upregulated pathways were related to energy metabolism and FAO. Specifically, the hepatic expression of PPARα, the master regulator of FAO, and its target genes was suppressed in NASH. This suppression was reversed in livers from mice treated with glycine or tripepetide glycine-glycine-leucine.

Subsequently, we applied an in vitro luciferase-based system to test whether glycine or tripepetide glycine-glycine-leucine directly activate PPARα.

### Methods

COS-1 and HepG2 cells were obtained from the American Type Culture Collection (ATCC) and cultured at 37° C. and 5% CO2 in Dulbecco’s Modified Eagle Medium (DMEM, Gibco) supplemented with 10% fetal bovine serum (FBS, Sigma-Aldrich) and 1% Penicillin-Streptomycin (Pen-Strep, Gibco). For luciferase assays, COS-1 cells were seeded in 96-well plates. At 60-70% confluence, transfection was performed using Lipofectamine 3000 (Invitrogen) with PPREx3-TK-luciferase, PPARα and Renilla constructs at 80 ng, 10 ng and 10 ng, respectively. 24 h post-transfection, cells were serum-starved and treated with 10 µM of the PPARα activator WY-14643 (Cayman Chemicals, 70730), 1 mM of glycine or tripepetide glycine-glycine-leucine or 10 µM of N-acyl amino acids for 24 h (Cayman Chemicals, C20:4-Gly 90051, C18:1-Leu 20064). Luciferase activity was assessed using the Dual-Luciferase Reporter Assay System (Promega) and normalized by Renilla.

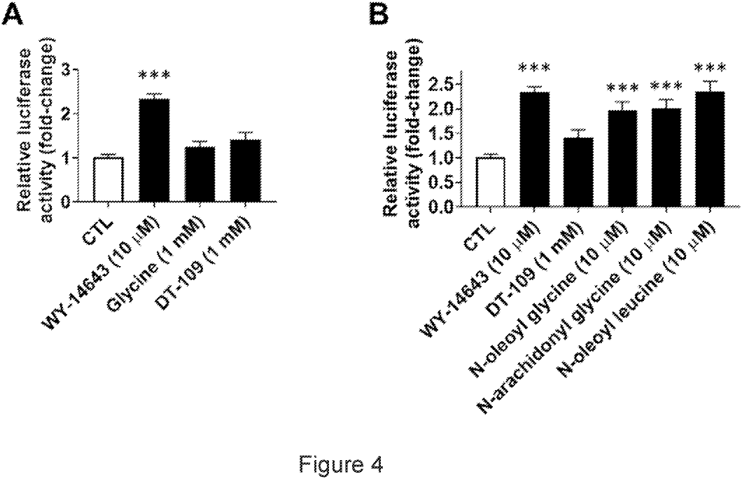

### Results

Whereas the known PPARα agonist, WY-14643, significantly increased the luciferase activity at 10 µM, neither glycine or tripepetide glycine-glycine-leucine showed a significant effect at concentrations of up to 1 mM (FIG. 4A). Next, we tested the activation of PPARα in response to N-acyl amino acids that were found to be higher in livers from mice fed CD or those fed NASH diet and treated with glycine or tripepetide glycine-glycine-leucine (FIGS. 1C-E). Similarly to WY-14643, 10 µM of N-oleoyl glycine (C18:1-Gly), N-arachidonoyl glycine (C20:4-Gly) or N-oleoyl leucine (C18:1-Leu) significantly increased luciferase activity (FIG. 4B).

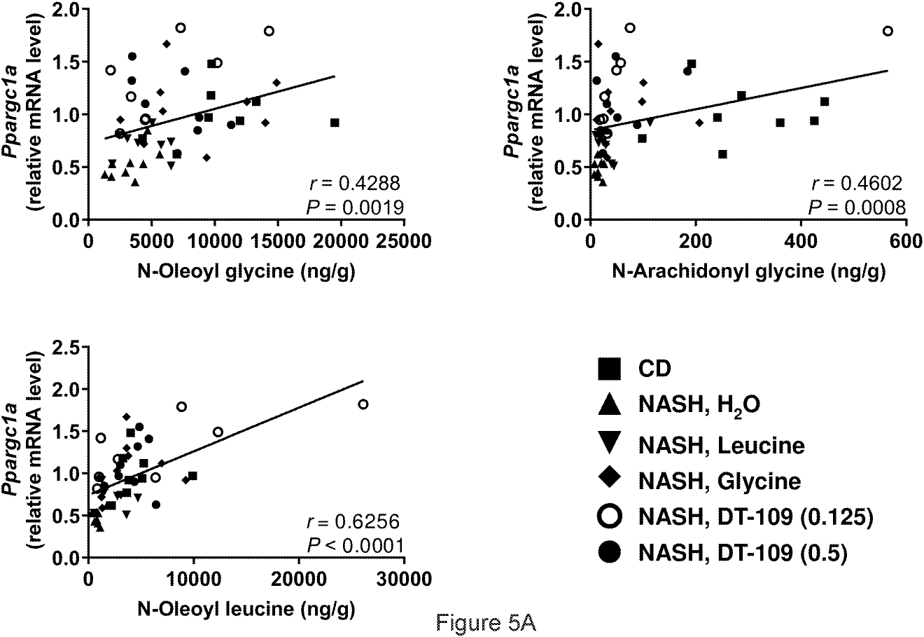

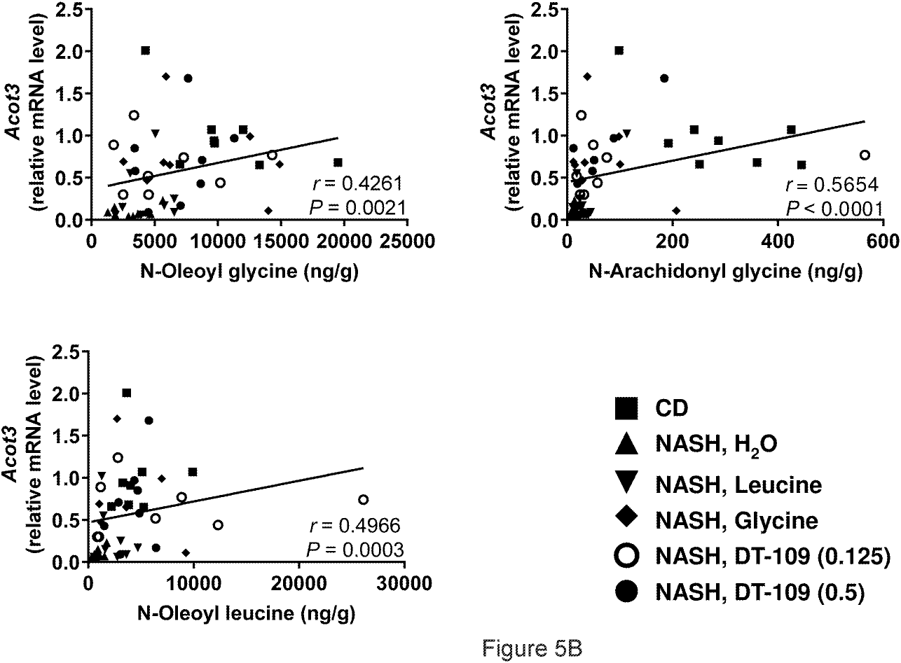

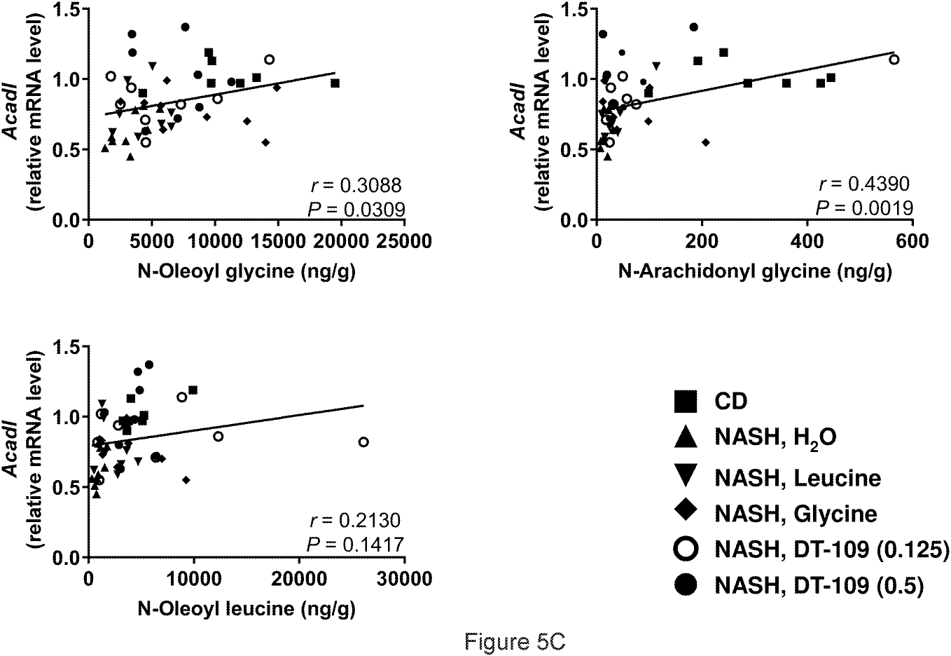

In vivo, the hepatic levels of N-acyl amino acids significantly and positively correlated with the expression of key PPARα target genes that play major roles in regulation of mitochondrial biogenesis and FAO, including peroxisome proliferative activated receptor, gamma, coactivator 1 alpha (Ppargc1a, FIG. 5A), acyl-CoA thioesterase 3 (Acot3, FIG. 5B) and acyl-CoA dehydrogenase, long chain (Acadl, FIG. 5C). Thus, N-acyl amino acids directly activate PPARα in vitro and are correlated with the expression of its key target genes in vivo.

### Example 4

### N-acyl Amino Acids Stimulate Lipid Utilization via Fatty Acid Β Oxidation

To assess a direct effect of N-acyl amino acids on lipid utilization via FAO, we applied Seahorse assays in HepG2 cells.

### Methods

HepG2 cells were obtained from the American Type Culture Collection (ATCC) and cultured at 37° C. and 5% CO2 in Dulbecco’s Modified Eagle Medium (DMEM, Gibco) supplemented with 10% fetal bovine serum (FBS, Sigma-Aldrich) and 1% Penicillin-Streptomycin (Pen-Strep, Gibco). Oxygen consumption rate (OCR) and dependency on FAO were assessed using a Seahorse XFe96 Analyzer (Agilent). HepG2 cells were seeded at 2.5x104 cells/well in XF96 cell culture microplates (Agilent). The next day, XFe96 sensor cartridges were hydrated in accordance with the manufacturer’s instructions. Cells were treated (port 1) with N-acyl amino acids (10 µM) or vehicle (EtOH), Etomoxir (Agilent, 6 µM, port 2) and finally with rotenone+antimycin A (R/A, Agilent, port 3).

For the TG biosynthesis and hydrolysis assays, HepG2 cells were seeded in 12-well plates. At 60-70% confluence, cells were treated with N-acyl amino acids (10 µM) or vehicle (EtOH) in serum-free medium supplemented with 0.1% BSA and stimulated for 3 h at 37° C. with [3H]-acetate (3.3 µCi/ml, ART 0202, American Radiolabeled Chemicals) to assess the rate TG biosynthesis. In some wells, the cells were washed twice with PBS ([3H]-acetate withdrawal) and incubated for additional 3 h with N-acyl amino acids (10 µM) or vehicle (EtOH) in serum-free medium supplemented with 0.1% BSA to assess the rate TG hydrolysis. At the end of the above incubation periods, cells were washed with twice with PBS. Cellular lipids were extracted using hexane (≥99%, Sigma-Aldrich 32293) and isopropanol (≥99.5%, Fisher Scientific A426-4) at a 3:2 ratio (v:v), and the hexane phase was left to evaporate for 48 h. Next, lipids were separated by thin layer chromatography (TLC) on silica gel plates (60 F254, M1057150001, Fisher Scientific) and developed in hexane / ether (≥99.9%, 309966, Sigma-Alrich) / acetic acid (≥99.7%, A38-212, Fisher Scientific) at a 130:30:1.5 ratio (v:v:v). TG spots were visualized by iodine vapor (using an appropriate standard for identification) and [3H]-labels were counted by a Tri-Carb 2810TR liquid scintillation analyzer (PerkinElmer). Data were normalized to protein levels and presented as count per minutes (CPM)/mg cell protein.

### Results

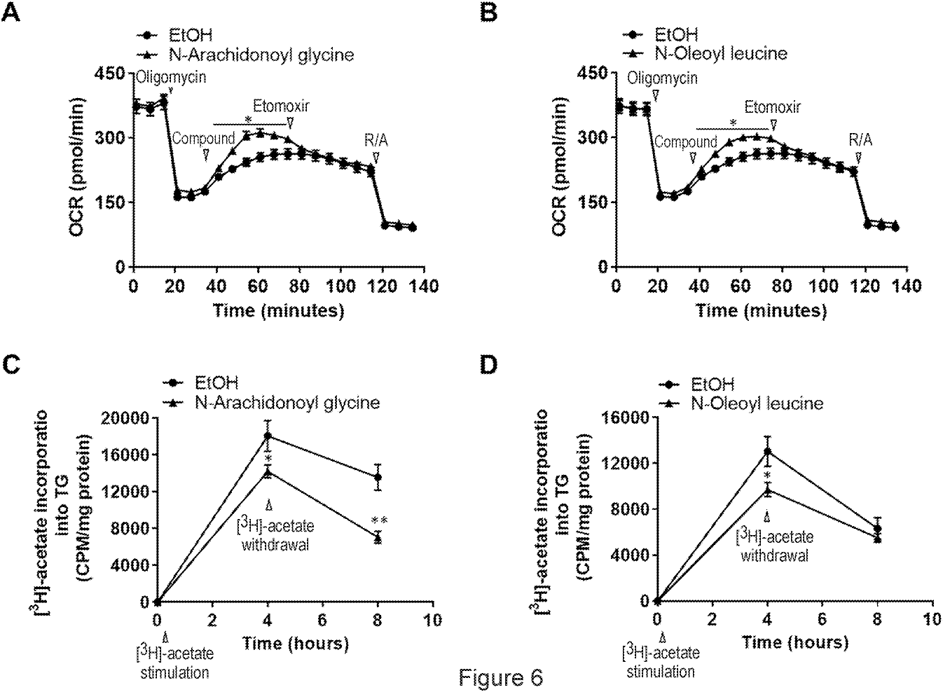

Acute stimulation with N-arachidonoyl glycine (C20:4-Gly) or N-oleoyl leucine (C18:1-Leu) significantly increased the oxygen consumption rate (OCR) by the cells which was attenuated by blocking FAO using etomoxir, an inhibitor of carnitine palmitoyltransferase-1 (CPT-1), a key player regulating essential steps of mitochondrial uptake of fatty acids and their subsequent β-oxidation (FIGS. 6A, B). Next, we assessed the rate of lipid biosynthesis and their hydrolysis by monitoring the incorporation of [3H]-labeled acetate into TG in the presence or absence of N-acyl amino acids. Both N-arachidonoyl glycine (C20:4-Gly) and N-oleoyl leucine (C18:1-Leu) attenuated the rate of TG biosynthesis, however, only N-arachidonoyl glycine (C20:4-Gly) significantly accelerated the rate of TG hydrolysis (FIGS. 6C, D). These results indicate that N-acyl amino acids, particularly N-arachidonoyl glycine (C20:4-Gly), directly stimulate lipid utilization via FAO highlighting their therapeutic potential against steatohepatitis, fibrosis and CVD.

### Example 5

### Treatment of NASH in Mice

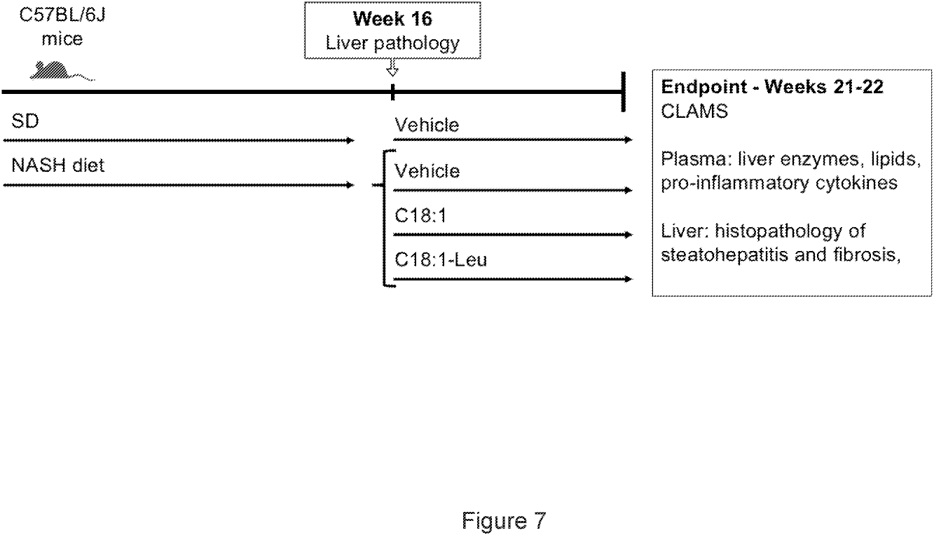

FIG. 7 shows the experimental design of a NASH study in mice.

### Methods

C57BL/6J mice were fed a standard diet (SD) or non-alcoholic steatohepatitis (NASH) diet for 16 weeks. After NASH confirmation, mice were randomized to receive 10 mg/kg/d (I.P.) N-oleoyl leucine (C18:1-Leu) or equivalent amounts of oleic acid (C18:1) or vehicle (EtOH) for an additional 6 weeks on the NASH diet. Control mice were fed the SD and administered vehicle.

### Results

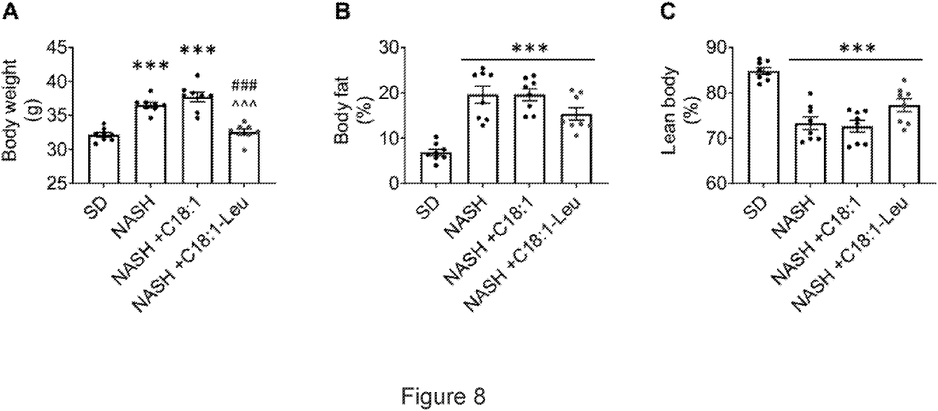

FIG. 8 demonstrates C18:1-Leu lowers body weight without affecting adiposity.

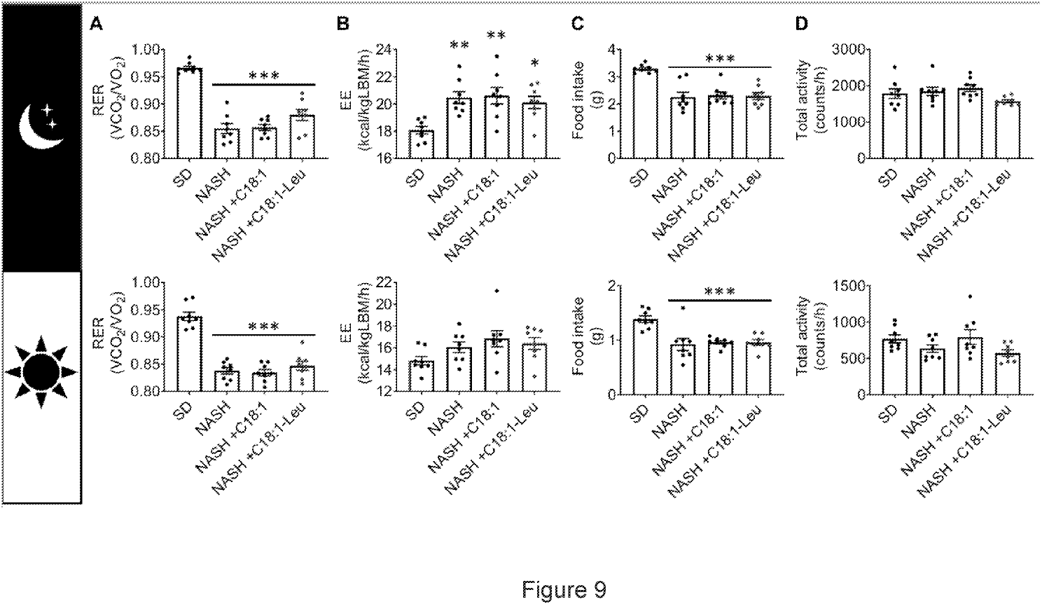

FIG. 9 demonstrates C18:1-Leu has no significant effect on systemic energy balance in NASH.

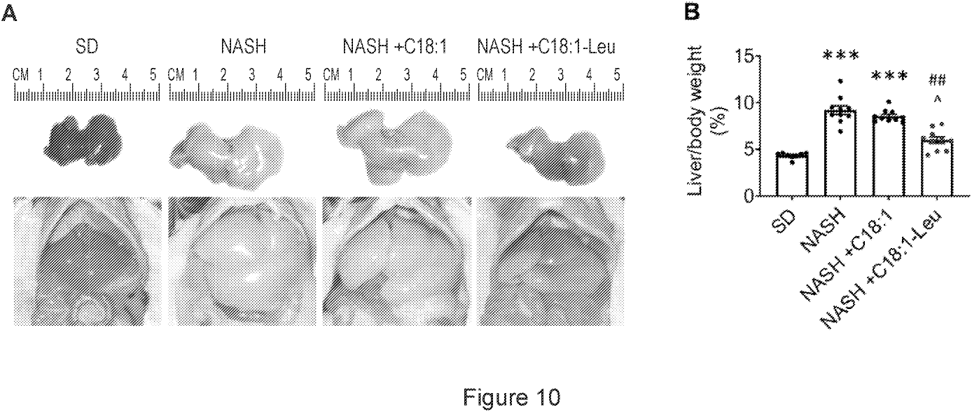

FIG. 10 demonstrates C18:1-Leu significantly lowers hepatomegaly.

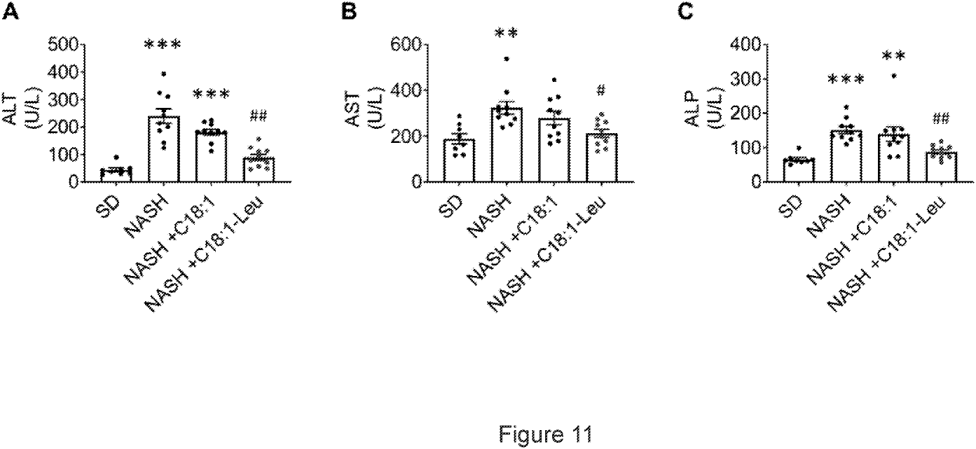

FIG. 11 demonstrates C18:1-Leu lowers circulating liver enzymes.

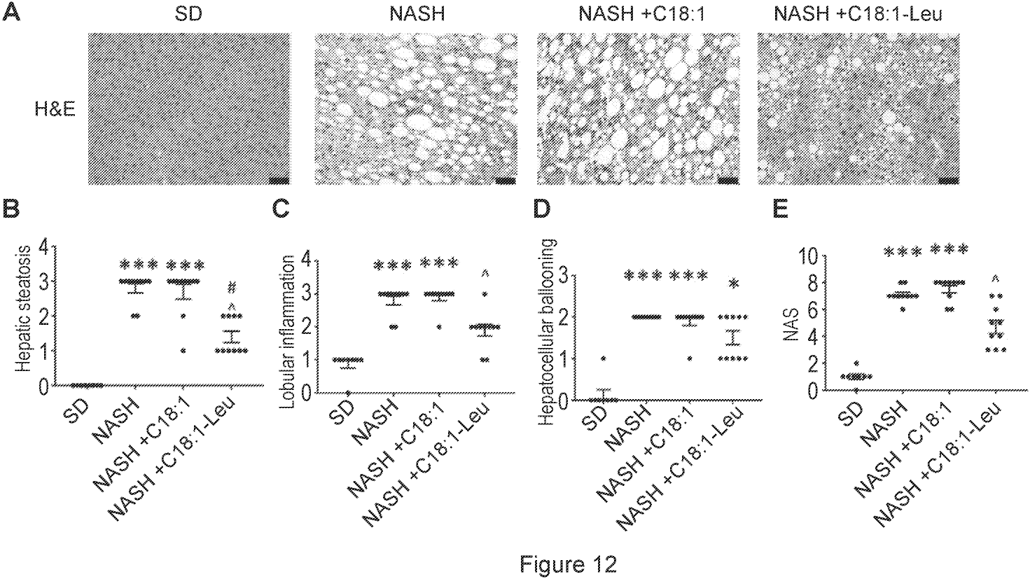

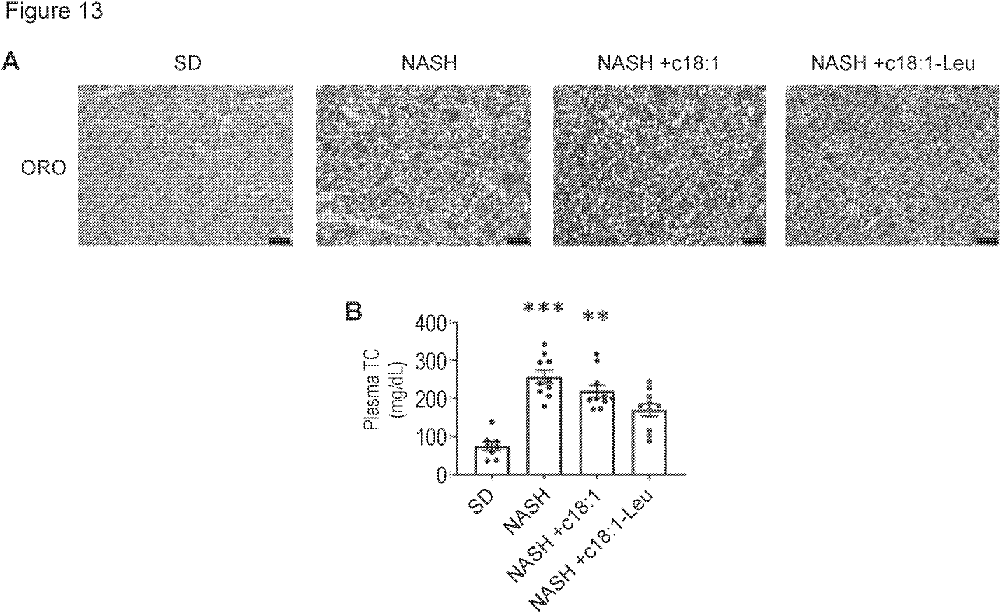

FIG. 12 demonstrates C18:1-Leu significantly lowers diet-induced NASH.

FIG. 13 demonstrates C18:1-Leu significantly lowers hepatic steatosis.

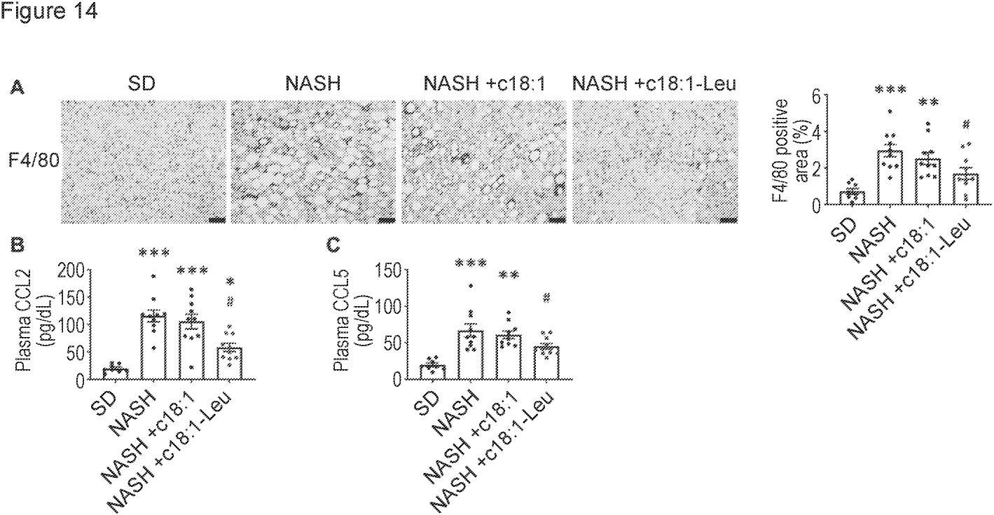

FIG. 14 demonstrates C18:1-Leu significantly lowers NASH diet-induced hepatic and systemic inflammation.

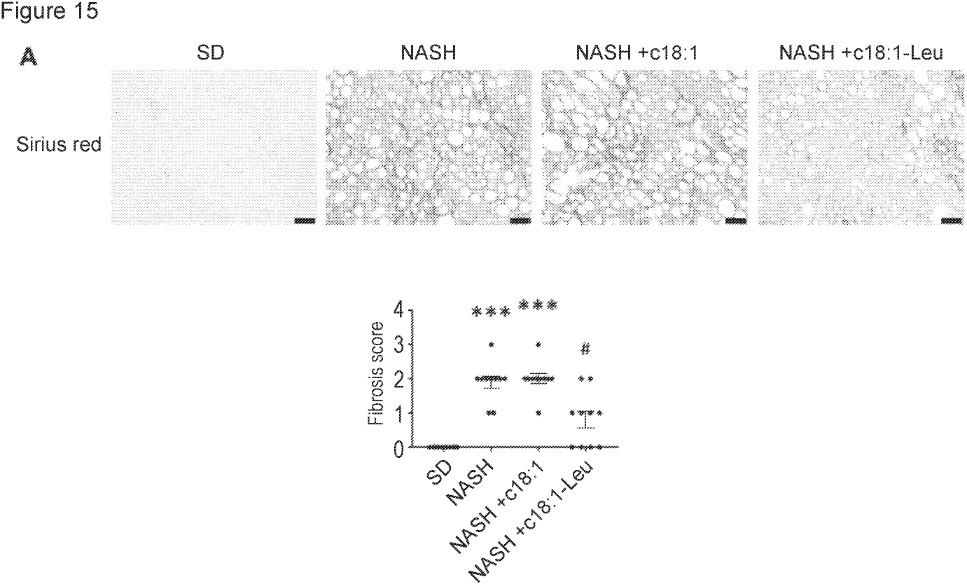

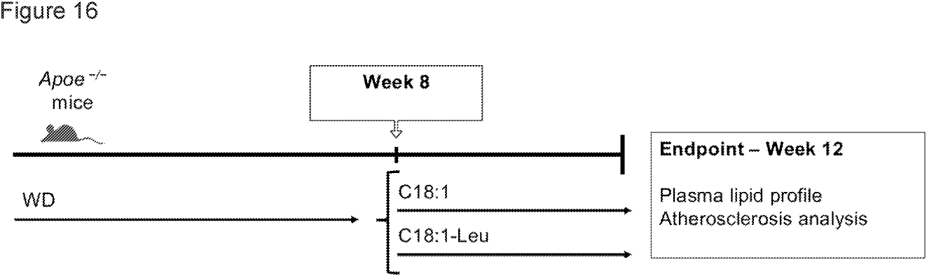

FIG. 15 demonstrates C18:1-Leu significantly lowers NASH diet-induced hepatic fibrosis.

### Example 6

### Treatment of Atherosclerosis in Mice

FIG. 16 shows the experimental design of an atherosclerosis study in mice.

### Methods

Apolipoprotein E-deficient (Apoe-′-) mice were fed a Western diet (WD) for 8 weeks. Mice were randomized to receive 7.5 mg/kg/d (I.P.) N-oleoyl leucine (C18:1-Leu) or equivalent amounts of oleic acid (C18:1) for an additional 4 weeks on the WD (n=10).

### Results

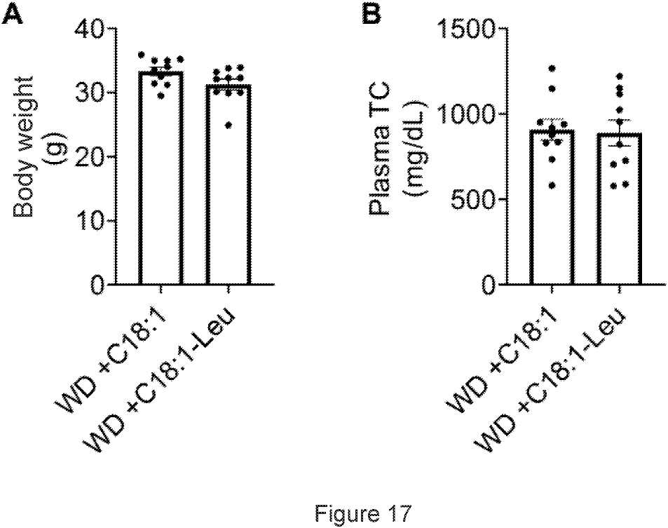

FIG. 17 demonstrates C18:1-Leu treatment had no significant effects on body weight and plasma cholesterol in atherosclerotic mice.

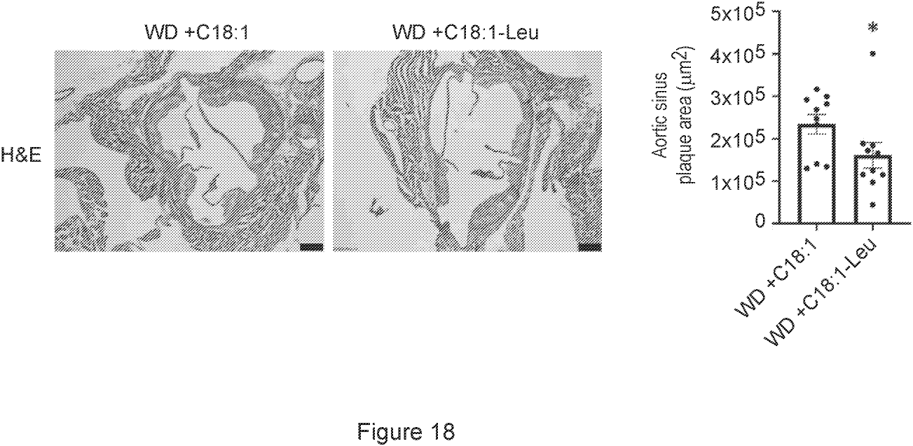

FIG. 18 demonstrates C18:1-Leu significantly lowers atherosclerotic plaque area.

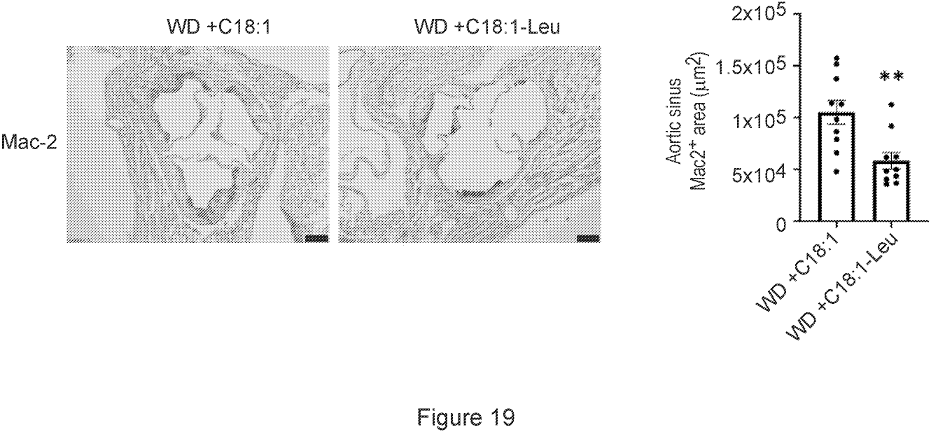

FIG. 19 demonstrates C18:1-Leu significantly lowers lesional macrophages.

While the present invention has been described in terms of various embodiments and examples, it is understood that variations and improvements will occur to those skilled in the art. Therefore, only such limitations as appear in the claims should be placed on the invention.

All documents referred to in this application are hereby incorporated by reference in their entirety with particular attention to the content for which they are referred.

We claim:

1. A method of treating a cardiovascular disease condition in a subject, comprising administering a pharmaceutically effective amount of at least one N-acyl amino acid product to the subject, wherein the N-acyl amino acid product has a fatty acid component and an amino acid component.

2. The method of claim 1 wherein the fatty acid component is a polyunsaturated fatty acid or a nitro fatty acid.

3. The method of claim 2 wherein the fatty acid component is an omega 3 fatty acid.

4. The method of claim 2 wherein the fatty acid component is a metabolite of an omega 3 fatty acid.

5. The method of claim 1 wherein the amino acid component is glycine, leucine or D-leucine.

6. The method of claim 1 wherein the amino acid component is a peptide.

7. The method of claim 6 wherein the amino acid component is glycine-glycine-leucine or glycine-glycine-D-leucine.

8. The method of claim 1 wherein at least N-arachidonoyl glycine or a pharmaceutically acceptable salt thereof is administered.

9. The method of claim 1 wherein at least N-oleoyl leucine or N-oleoyl D-leucine, or a pharmaceutically acceptable salt thereof is administered.

10. The method of claim 1 wherein at least N-arachidonoyl glycine-glycine-leucine, N-oleoyl glycine-glycine-leucine, N-arachidonoyl glycine-glycine-D-leucine, or N-oleoyl glycine-glycine-D-leucine, or a pharmaceutically acceptable salt thereof is administered.

11. The method of claim 1 wherein the cardiovascular disease condition is coronary heart disease, cerebrovascular disease, peripheral arterial disease, rheumatic heart disease, congenital heart disease, aortic aneurysm or deep vein thrombosis and pulmonary embolism.

12. The method of claim 1 wherein the treating results in the subject in one or more of: decreased atherosclerotic plaques, increased cardiac function, reduced myocardial hypertrophy, decreased blood pressure, decreased inflammatory status and decreased aortic diameter.

13. The method of claim 1 wherein the treating mitigates one or more of heart failure, decompensation, myocardial infarction and aneurysms.

14. A method of treating steatohepatitis in a subject, comprising administering a pharmaceutically effective amount of at least one N-acyl amino acid product to the subject, wherein the N-acyl amino acid product has a fatty acid component and an amino acid component.

15. The method of claim 14 wherein the fatty acid component is a polyunsaturated fatty acid or a nitro fatty acid.

16. The method of claim 15 wherein the fatty acid component is an omega 3 fatty acid.

17. The method of claim 15 wherein the fatty acid component is a metabolite of an omega 3 fatty acid.

18. The method of claim 14 wherein the amino acid component is glycine, leucine or D-leucine.

19. The method of claim 14 wherein the amino acid component is a peptide.

20. The method of claim 19 wherein the amino acid component is glycine-glycine-leucine or glycine-glycine-D-leucine.

21. The method of claim 14 wherein at least N-arachidonoyl glycine or a pharmaceutically acceptable salt thereof is administered.

22. The method of claim 14 wherein at least N-oleoyl leucine or N-oleoyl D-leucine, or a pharmaceutically acceptable salt thereof is administered.

23. The method of claim 14 wherein at least N-arachidonoyl glycine-glycine-leucine, N-oleoyl glycine-glycine-leucine, N-arachidonoyl glycine-glycine-D-leucine, or N-oleoyl glycine-glycine-D-leucine, or a pharmaceutically acceptable salt thereof is administered.

24. The method of claim 14 wherein the steatohepatitis is non-alcoholic steatohepatitis, alcoholic liver disease or alcoholic steatohepatitis.

25. The method of claim 14 wherein the treating results in the subject in decreased liver fat, decreased inflammatory status, decreased injured hepatocytes and decreased atherosclerotic plaques.

26. The method of claim 14 wherein the treating mitigates one or more of cirrhosis and hepatocellular carcinoma.

27. A method of treating fibrosis in a subject, comprising administering a pharmaceutically effective amount of an N-acyl amino acid product to the subject, wherein the N-acyl amino acid product has a fatty acid component and an amino acid component.

28. The method of claim 27 wherein the fatty acid component is a polyunsaturated fatty acid or a nitro fatty acid.

29. The method of claim 28 wherein the fatty acid component is an omega 3 fatty acid.

30. The method of claim 28 wherein the fatty acid component is a metabolite of an omega 3 fatty acid.

31. The method of claim 27 wherein the amino acid component is glycine, leucine or D-leucine.

32. The method of claim 27 wherein the amino acid component is a peptide.

33. The method of claim 32 wherein the amino acid component is glycine-glycine-leucine or glycine-glycine-D-leucine.

34. The method of claim 27 wherein at least N-arachidonoyl glycine or a pharmaceutically acceptable salt thereof is administered.

35. The method of claim 27 wherein at least N-oleoyl leucine or N-oleoyl D-leucine, or a pharmaceutically acceptable salt thereof is administered.

36. The method of claim 27 wherein at least N-arachidonoyl glycine-glycine-leucine, N-oleoyl glycine-glycine-leucine, N-arachidonoyl glycine-glycine-D-leucine, or N-oleoyl glycine-glycine-D-leucine, or a pharmaceutically acceptable salt thereof is administered.

37. The method of claim 27 wherein the treating results in the subject in a reduction in collagen in the liver, heart, lungs, kidneys, skin or adipose tissue.

38. The method of claim 27 wherein the treating results in the subject in a reduction in collagen in the bile duct or gallbladder.

39. A pharmaceutical composition comprising (a) an excipient and (b) N-arachidonoyl glycine or a pharmaceutically acceptable salt thereof.

40. A pharmaceutical composition comprising (a) an excipient and (b) N-oleoyl leucine or a pharmaceutically acceptable salt thereof.

41. A pharmaceutical composition comprising (a) an excipient and (b) N-oleoyl D-leucine or a pharmaceutically acceptable salt thereof.

42. A pharmaceutical composition comprising (a) an excipient and (b) one or more of N-arachidonoyl glycine, N-oleoyl leucine and N-oleoyl D-leucine, or pharmaceutically acceptable salts thereof.

43. A pharmaceutical composition comprising (a) an excipient and (b) N-arachidonoyl glycine-glycine-leucine or a pharmaceutically acceptable salt thereof.

44. A pharmaceutical composition comprising (a) an excipient and (b) N-oleoyl glycine-glycine-leucine or a pharmaceutically acceptable salt thereof.

45. A pharmaceutical composition comprising (a) an excipient and (b) N-arachidonoyl glycine-glycine-D-leucine or a pharmaceutically acceptable salt thereof.

46. A pharmaceutical composition comprising (a) an excipient and (b) N-oleoyl glycine-glycine-D-leucine or a pharmaceutically acceptable salt thereof.

47. A pharmaceutical composition comprising (a) an excipient and (b) at least two of arachidonoyl glycine-glycine-leucine, N-oleoyl glycine-glycine-leucine, N-arachidonoyl glycine-glycine-D-leucine, or N-oleoyl glycine-glycine-D-leucine, or pharmaceutically acceptable salts thereof.

48. A pharmaceutical composition comprising (a) an excipient and (b) at least two of N-arachidonoyl glycine, N-oleoyl leucine, N-oleoyl D-leucine, N-arachidonoyl glycine-glycine-leucine, N-oleoyl glycine-glycine-leucine, N-arachidonoyl glycine-glycine-D-leucine, or N-oleoyl glycine-glycine-D-leucine, or pharmaceutically acceptable salts thereof.

49. A method of diagnosing a disease condition in a subject, comprising detecting at least one N-acyl amino acid in the subject,
wherein the N-acyl amino acid is at least one of N-arachidonoyl glycine, N-oleoyl leucine and N-oleoyl D-leucine, and
wherein the level of the N-acyl amino acid is negatively associated with the disease condition.

50. The method of claim 49 wherein the disease condition is a cardiovascular disease condition.

51. The method of claim 49 wherein the disease condition is steatohepatitis.

52. The method of claim 49 wherein the disease condition is fibrosis.
